(19) United States
(12) Patent Application Publication
(10) Pub. No.: US 2020/0332363 A1
(43) Pub. Date: Oct. 22, 2020

(54) METHODS FOR PREDICTING RISK OF RECURRENCE AND/OR METASTASIS IN SOFT TISSUE SARCOMA

(71) Applicants: Robert Willis Cook, Friendswood, TX (US); Weiwei Shan, Friendswood, TX (US); Derek Maetzold, Friendswood, TX (US); CASTLE BIOSCIENCES, INC., Friendswood, TX (US)

(72) Inventors: Robert Willis COOK, Friendswood, TX (US); Weiwei SHAN, Friendswood, TX (US); Derek MAETZOLD, Friendswood, TX (US)

(73) Assignee: Castke Bioscience, Inc., Friendswood, TX (US)

(21) Appl. No.: 16/306,018

(22) PCT Filed: Jun. 5, 2017
(86) PCT No.: PCT/US2017/036015

§ 371 Date: Nov. 30, 2018

Related U.S. Application Data
(60) Provisional application No. 62/345,488, filed on Jun. 3, 2016.
(60) Provisional application No. 62/345,475, filed on Jun. 3, 2016.

(51) Int. Cl. C12Q 1/6886

(52) U.S. Cl. CPC C12Q 1/6886; C12Q 2600/158; C12Q 2600/118

(57) ABSTRACT

The disclosure related to the development of a gene expression profile to predict soft tissue sarcoma (STS) recurrence, distant metastasis, or both. Analyses identified a 36-gene gene expression profile able to accurately predict risk in a cohort of soft tissue sarcoma tumors independent of histologic and pathologic grade. This discovery offers an opportunity to enhance current staging of STS to identify patients who have a higher risk of recurring, distant metastasis, or both.

### CROSS REFERENCE

This application claims priority to U.S. Provisional Patent Application No. 62/345,475, filed Jun. 3, 2016, and to U.S. Provisional Patent Application No. 62/345,488, filed Jun. 3, 2016, the disclosures of each which are incorporated by reference herein in their entirety.

### BACKGROUND

Malignant soft tissue sarcomas (STS) are rare mesenchymal tumors originating from soft tissues, including fat, muscle, nerve (and nerve sheath), blood vessel wall and connective tissues. STSs account for approximately 12,000 cancer cases in the U.S. each year, and cause roughly 4,700 deaths annually. However, the reported incidence of STS may be underestimated, due to previous exclusion of gastrointestinal stromal tumors (GISTs) from the STS category. Classification of STS subtypes generally follows the rules set out by the Federation Francaise des Centres de Lutte Contre le cancer (FNCLCC). More than 50 different STS histotypes have been discovered, the most common being undifferentiated pleomorphic sarcoma (UPS; previously known as malignant fibrous histiocytoma, MFH), GISTs, liposarcoma, leiomyosarcoma, synovial sarcoma, and malignant peripheral nerve sheath. UPS and rabdomyosarcoma (RMS) are the most common STS subtypes seen in adults and children (and adolescents), respectively. Sarcoma is associated with a higher morbidity and mortality rate in adults compared to children.

Physicians have largely relied on conventional clinicopathologic factors, such as tumor size, location, degree of differentiation, and histotype, to assess the risk associated with primary STS tumors. However, clinical features alone are not sufficient to accurately stratify tumors into distinct risk groups. Recent efforts have focused on identifying genetic markers to differentiate between tumors with different risk profiles. Chibon et al. have reported the discovery of a 67-probe microarray-based genetic signature able to predict risk of metastasis for patients with both non-translocation (LMS, UPS, dedifferentiated liposarcoma) and translocation-specific (synovial sarcoma) type sarcomas (Chibon et al. (2010) Nat Med, 16(7):781-87). Genomic profiling of LMS and UPS have also identified specific genomic losses and gains associated with risk for metastasis. However, a clinically validated biomarker test able to accurately prognosticate STS, particularly the non-translocation type with aggressive clinical behavior, is not yet available.

### SUMMARY OF THE INVENTION

There is a need in the art for an accurate and objective method of predicting which tumors possess aggressive metastatic potential. Development of an accurate molecular footprint, such as the gene expression profile encompassed by the invention disclosed herein, by which STS metastatic risk could be assessed from primary tumor tissue, would be a significant advance forward for the field. Inaccurate prognosis for metastatic risk has profound effects upon patients, including over-treatment of low risk patients that includes enhanced surveillance, nodal surgery, and chemotherapy, and under-treatment of high risk patients who are likely to experience recurrence of disease.

In an aspect, the disclosure relates to a method for predicting risk of local recurrence, distant metastasis, or both, in a patient with a primary soft tissue sarcomas (STS) tumor, the method comprising: (a) obtaining a STS tumor sample from the patient and isolating mRNA from the sample; (b) determining the expression level of at least 10 genes in a gene set; wherein the at least ten genes in the gene set are selected from: ABCB1, ABCC1, ABCG2, ACTB, ALAS1, ANLN, ANXA1, AQP3, BAX, Bcl2, Bcl2L/Bcl-xl, BIRC5, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPA5, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT; (c) comparing the expression levels of the at least 10 genes in the gene set from the STS tumor sample to the expression levels of the at least 10 genes in the gene set from a predictive training set to generate a probability score of the risk of local recurrence, distant metastasis, or both, and (d) providing an indication as to whether the STS tumor has a low risk to a high risk of local recurrence, distant metastasis, or both, based on the probability score generated in step (c). In certain embodiments of the method, the gene set comprises the genes ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN.

In another aspect, the disclosure relates to a method for treating a patient with a primary soft tissue sarcomas (STS) tumor, the method comprising: (a) obtaining a diagnosis identifying a risk of local recurrence, distant metastasis, or both, in a STS tumor sample from the patient, wherein the diagnosis was obtained by: (1) determining the expression level of at least 10 genes in a gene set; wherein the at least 10 genes in the gene set are selected from: ABCB1, ABCC1, ABCG2, ACTB, ALAS1, ANLN, ANXA1, AQP3, BAX, Bcl2, Bcl2L/Bcl-xl, BIRC5, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPA5, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT; (2) comparing the expression levels of the at least 10 genes in the gene set from the STS tumor sample to the expression levels of the at least 10 genes in the gene set from a predictive training set to generate a probability score of the risk of local recurrence, distant metastasis, or both, and; (3) providing an indication as to whether the STS tumor has a low risk to a high risk of local recurrence, distant metastasis, or both, based on the probability score generated in step (2); and (4) identifying that the STS tumor has a high risk of local recurrence, distant metastasis, or both, based on the probability score and diagnosing the STS tumor as having a high risk of local recurrence, distant metastasis, or both; (b) administering to the patient an aggressive treatment when the determination is made in the affirmative that the patient has a STS tumor with a high risk of local recurrence, distant metastasis, or both. In certain embodiments of the method, the gene set comprises the genes ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN.

In yet another aspect, the disclosure relates to a method of treating a patient with a primary soft tissue sarcoma (STS) tumor, the method comprising administering an aggressive cancer treatment regimen to the patient, wherein the patient has a STS tumor with a probability score of between 0.500 and 1.00 as generated by comparing the expression levels of at least 10 genes selected from ABCB1, ABCC1, ABCG2, ACTB, ALAS1, ANLN, ANXA1, AQP3, BAX, Bcl12, Bcl2L/Bcl-xl, BIRCS, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPAS, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT from the STS tumor with the expression levels of the same at least ten genes selected from ABCB1, ABCC1, ABCG2, ACTB, ALAS1, ANLN, ANXA1, AQP3, BAX, Bcl2, Bcl2L/Bcl-xl, BIRCS, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPAS, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT from a predictive training set. In certain embodiments of the method, the probability score is determined by a bimodal, two-class analysis, wherein a patient having a value of between 0 and 0.499 is designated as class 1 with a low risk of local recurrence, distant metastasis, or both, and a patient having a value of between 0.500 and 1.00 is designated as class 2 with an increased risk of local recurrence, distant metastasis, or both. In an embodiment of the method, the gene set comprises the genes ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN.

In an additional aspect, the disclosure relates to a kit comprising primer pairs suitable for the detection and quantification of nucleic acid expression of at least ten genes selected from: ABCB1, ABCC1, ABCG2, ACTB, ALAS1, ANLN, ANXA1, AQP3, BAX, Bcl2, Bcl2L/Bcl-xl, BIRCS, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPAS, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT. In an embodiment of the kit, the primer pairs suitable for the detection and quantification of nucleic acid expression of at least ten genes are primer pairs for: ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN. In certain embodiments of the kit, the primer pairs further comprise primer pairs for ABCC1, ACTB, RelA, STAT5B, and YY1AP1.

This disclosure provides a more objective method that more accurately predicts which STS tumors display aggressive metastatic activity and result in decreased patient disease-related survival. Development of an accurate molecular footprint, such as the gene expression profile assay encompassed by the invention disclosed herein, by which STS metastatic risk and patient disease-specific survival could be assessed from primary tumor tissue would be a significant advance forward for the field leading to decreased loss of life, less patient suffering, more efficient treatments and use of resources.

Specific embodiments of the invention will become evident from the following more detailed description of certain embodiments and the claims.

### BRIEF DESCRIPTION OF THE DRAWINGS

The disclosed exemplary aspects have other advantages and features which will be more readily apparent from the detailed description, the appended claims, and the accompanying figures. A brief description of the drawings is below.

FIG. 1A-FIG. 1C show that the 36-gene gene expression profile predicts risk for disease recurrence in the current cohort of 63 primary STS cases. Averaged AUC curves generated by 10-fold (FIG. 1A), 5-fold (FIG. 1B), and leave-3 (FIG. 1C) hold-out cross validation with 50 iterations for each method.

FIG. 2A-FIG. 2C show that the 36-gene gene expression profile predicts class 1 (low risk) and class 2 (high risk) patients with highly stratified 5-year relapse-free survival (RFS) (FIG. 2A; p<0.0001), 5-year metastasis-free survival (MFS) (FIG. 2B; p<0.001), and disease-specific survival (DSS) (FIG. 2C; p<0.09).

FIG. 3A-FIG. 3C show that the 36-gene gene expression profile predicts risk class A (low risk) and class C (high risk), and establishment an intermediate risk class B for probability scores RFS (FIG. 3A; p<0.0001), MFS (FIG. 3B; p=0.003), and DSS (FIG. 3C; p=0.1).

FIG. 4A-FIG. 4F show that the 36-gene gene expression profile predicted risk of class 1 and risk class 2 had significantly more stratified RFS as compared to patients' clinical factors in Kaplan-Meier survival. Kaplan-Meier survival analysis was performed to assess RFS in patient groups stratified according to the 36-gene GEP prediction (FIG. 4A), and conventional patho-clinical factors of STS of prognostic value, including diagnostic stage (FIG. 4B), tumor differentiation grade (FIG. 4C), location of primary tumor (extremity vs non-extremity) (FIG. 4D), size of tumor (5 cm cutoff) (FIG. 4E), and tumor histotype (LMS, UPS, or others) (FIG. 4F).

FIG. 5A-FIG. 5F show that the 36-gene gene expression profile predicted risk of class 1 and risk class 2 had significantly more stratified MFS as compared to patients' clinical factors in Kaplan-Meier. Kaplan-Meier analyses were performed to assess MFS in patient groups stratified according to the 36-gene GEP prediction (FIG. 5A), and conventional patho-clinical factors of STS of prognostic value, including diagnostic stage (FIG. 5B), tumor differentiation grade (FIG. 5C), location of primary tumor (extremity vs non-extremity) (FIG. 5D), size of tumor (5 cm cutoff) (FIG. 5E), and tumor histotype (LMS, UPS, or others) (FIG. 5F).

### DETAILED DESCRIPTION OF THE INVENTION

Unless otherwise defined, all technical and scientific terms used herein have the same meaning as commonly understood by one of ordinary skill in the art to which the invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting. Other features and advantages of the invention will be apparent from the following detailed description. Applicants reserve the right to alternatively claim any disclosed invention using the transitional phrase “comprising,” “consisting essentially of,” or “consisting of,” according to standard practice in patent law.

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide”, “nucleotide”, “oligonucleotide”, and “nucleic acid” can be used interchangeably to refer to nucleic acid comprising DNA, cDNA, RNA, derivatives thereof, or combinations thereof.

This disclosure provides a more objective method that more accurately predicts which soft tissue sarcoma (STS) tumors display aggressive metastatic activity and result in decreased patient disease-related survival. Development of an accurate molecular footprint, such as the gene expression profile encompassed by the invention disclosed herein, by which STS metastatic risk and patient disease-specific survival could be assessed from primary tumor tissue would be a significant advance forward for the field leading to decreased loss of life, less patient suffering, more efficient treatments and use of resources.

In an aspect, the disclosure relates to a method for predicting risk of local recurrence, distant metastasis, or both, in a patient with a primary soft tissue sarcomas (STS) tumor, the method comprising: (a) obtaining a STS tumor sample from the patient and isolating mRNA from the sample; (b) determining the expression level of at least 10 genes in a gene set; wherein the at least ten genes in the gene set are selected from: ABCB1, ABCC1, ABCG2, ACTB, ALAS1, ANLN, ANXA1, AQP3, BAX, Bcl2, Bcl2L/Bcl-xl, BIRC5, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPA5, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT; (c) comparing the expression levels of the at least 10 genes in the gene set from the STS tumor sample to the expression levels of the at least 10 genes in the gene set from a predictive training set to generate a probability score of the risk of local recurrence, distant metastasis, or both, and (d) providing an indication as to whether the STS tumor has a low risk to a high risk of local recurrence, distant metastasis, or both, based on the probability score generated in step (c). In certain embodiments of the method, the gene set comprises the genes ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN.

In another aspect, the disclosure relates to a method for treating a patient with a primary soft tissue sarcomas (STS) tumor, the method comprising: (a) obtaining a diagnosis identifying a risk of local recurrence, distant metastasis, or both, in a STS tumor sample from the patient, wherein the diagnosis was obtained by: (1) determining the expression level of at least 10 genes in a gene set; wherein the at least 10 genes in the gene set are selected from: ABCB1, ABCC1, ABCG2, ACTB, ALAS1, ANLN, ANXA1, AQP3, BAX, Bcl2, Bcl2L/Bcl-xl, BIRC5, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPA5, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT; (2) comparing the expression levels of the at least 10 genes in the gene set from the STS tumor sample to the expression levels of the at least 10 genes in the gene set from a predictive training set to generate a probability score of the risk of local recurrence, distant metastasis, or both, and; (3) providing an indication as to whether the STS tumor has a low risk to a high risk of local recurrence, distant metastasis, or both, based on the probability score generated in step (2); and (4) identifying that the STS tumor has a high risk of local recurrence, distant metastasis, or both, based on the probability score and diagnosing the STS tumor as having a high risk of local recurrence, distant metastasis, or both; (b) administering to the patient an aggressive treatment when the determination is made in the affirmative that the patient has a STS tumor with a high risk of local recurrence, distant metastasis, or both. In certain embodiments of the method, the gene set comprises the genes ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN.

In yet another aspect, the disclosure relates to a method of treating a patient with a primary soft tissue sarcoma (STS) tumor, the method comprising administering an aggressive cancer treatment regimen to the patient, wherein the patient has a STS tumor with a probability score of between 0.500 and 1.00 as generated by comparing the expression levels of at least 10 genes selected from ABCB1, ABCC1, ABCG2, ACTB, ALAS1, ANLN, ANXA1, AQP3, BAX, Bcl2, Bcl2L/Bcl-xl, BIRCS, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPAS, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT from the STS tumor with the expression levels of the same at least ten genes selected from ABCB1, ABCC1, ABCG2, ACTB, ALAS1, ANLN, ANXA1, AQP3, BAX, Bcl2, Bcl2L/Bcl-xl, BIRCS, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPAS, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT from a predictive training set. In certain embodiments of the method, the probability score is determined by a bimodal, two-class analysis, wherein a patient having a value of between 0 and 0.499 is designated as class 1 with a low risk of local recurrence, distant metastasis, or both, and a patient having a value of between 0.500 and 1.00 is designated as class 2 with an increased risk of local recurrence, distant metastasis, or both. In an embodiment of the method, the gene set comprises the genes ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN.

In an embodiment, the risk of recurrence or metastasis for the primary soft tissue sarcoma tumor is classified from a low risk to a high risk (for example, the tumor has a graduated risk from low risk to high risk or high risk to low risk of local recurrence, locoregional recurrence, or distant metastasis). In other embodiments, low risk refers to a 5-yr relapse-free survival rate, a 5-yr metastasis free survival rate, or a 5-yr disease specific survival rate of greater than 50%, 55%, 60%, 65%, 70%, 75%, 80%, 85%, 90%, 95%, or more, and high risk refers to a 5-yr relapse-free survival rate, a 5-yr metastasis free survival rate, or a 5-yr disease specific survival rate of less than 50%, 45%, 40%, 35%, 30%, 25%, 20%, 15%, 10%, 5%, or less.

In certain embodiments, class 1 indicates that the tumor is at a low risk of local recurrence, or distant metastasis, or both, and class 2 indicates that the tumor is at a high risk of local recurrence, or distant metastasis, or both. Class A indicates that the tumor is at a low risk of local recurrence, or distant metastasis, or both, class B indicates that the tumor is at an intermediate risk of local recurrence, or distant metastasis, or both, and class C indicates that the tumor is at a high risk of local recurrence, or distant metastasis, or both.

As used herein, the term “metastasis” is defined as recurrence or disease progression that may occur locally, regionally (such as nodal metastasis), or distally (such as distant metastasis to the brain, lung and other tissues). Class 1 or class 2 of metastasis as defined herein includes low-risk (class 1) or high-risk (class 2) of metastasis according to any of the statistical methods disclosed herein. Class A, Class B, or Class C of metastasis as defined herein includes low-risk (class A), intermediated risk (class B) or high-risk (class C) of metastasis according to any of the statistical methods disclosed herein. The term “distant metastasis” as used herein, refers to metastases from a primary STS tumor that are disseminated widely. Patients with distant metastases require aggressive treatments, which can eradicate metastatic sarcoma, prolong life and cure some patients.

As used herein, the terms “locoregional recurrence” and “local recurrence” can be used interchangeably and refer to cancer cells that have spread to tissue immediately surrounding the primary STS tumor or were not completely ablated or removed by previous treatment or surgical resection. Locoregional recurrences are typically resistant to chemotherapy and radiation therapy. Locoregional recurrence can be difficult to control and/or treat if: (1) the primary STS is located or involves a vital organ or structure that limits the potential for treatment; (2) recurrence after surgery or other therapy occurs, because while likely not a result from metastasis, high rates of recurrence indicate an advanced STS tumor; and (3) presence of lymph node metastases, while rare in STS, indicate advanced disease.

In some embodiments, the methods described herein can comprise determining that the STS tumor has an increased risk of metastasis or decreased overall survival by combining with clinical staging factors recommended by the American Joint Committee on Cancer (AJCC) to stage the primary STS tumor, or other histological features associated with risk of STS tumor metastasis or disease-related death.

As used herein, the terms “soft tissue sarcoma” or “STS” refer to any primary STS lesion, regardless of tumor size, in patients without clinical or histologic evidence of regional or distant metastatic disease and which may be obtained through a variety of sampling methods such as core needle biopsy, incisional biopsy, endoscope ultrasound (EUS) guided-fine needle aspirate (FNA) biopsy, percutaneous biopsy, punch biopsy, surgical excision, and other means of extracting RNA from the primary STS lesion. A sarcoma is a type of cancer that develops from certain tissues, like bone or muscle. Bone and soft tissue sarcomas are the main types of sarcoma. Soft tissue sarcomas can develop from soft tissues like fat, muscle, nerves, fibrous tissues, blood vessels, or deep skin tissues. They can be found in any part of the body. Most of them develop in the arms or legs. They can also be found in the trunk, head and neck area, internal organs, and the area in back of the abdominal cavity. Sarcomas are not common tumors. Examples of soft tissue sarcomas can include, but are not limited to: adult fibrosarcoma, alveolar soft-part sarcoma, angiosarcoma (including hemangiosarcoma and lymphangiosarcoma), clear cell sarcoma, desmoplastic small round cell tumor, epithelioid sarcoma, fibromyxoid sarcoma, low-grade gastrointestinal stromal tumor (GIST) (this is a type of sarcoma that develops in the digestive tract), kaposi sarcoma (this is a type of sarcoma that develops from the cells lining lymph or blood vessels), liposarcoma (including dedifferentiated, myxoid, and pleomorphic liposarcomas), leiomyosarcoma, malignant mesenchymoma, malignant peripheral nerve sheath tumors (including neurofibrosarcomas, neurogenic sarcomas, and malignant schwannomas), myxofibrosarcoma, low-grade rhabdomyosarcoma (this is the most common type of soft tissue sarcoma seen in children), synovial sarcoma, undifferentiated pleomorphic sarcoma (previously known as malignant fibrous histiocytoma or MFH). Morphologic and histologic characteristics of a few common STS are listed in Table 1 below.

TABLE 1
Common STS histotypes.

| Subtype | Epidemiology | Presentation | Pathology and genetics |
|---|---|---|---|
| undifferentiated | Most common STS in | Occurs most commonly in | High cellularity, marked |
| pleomorphic sarcoma | adults. Occurs more | the extremities and | nuclear pleomorphism, |
| (UPS, previously | often in Caucasians than | retroperitoneum | abundant mitosis |
| MFH) | in African or Asian | | |
| | descents | | |
| Gastrointestinal | Most common | 70% occurs in the stomach, | 85% harbor mutations in the |
| stromal tumor (GIST) | mesenchymal tumor of | 20% in the small intestine | KIT oncogene, 10% in |
| | the GI tract. | and <10% in the esophagus. | PDGFRA, a few in BRAF |
| | | GISTs have a lower | |
| | | malignant potential than | |
| | | other GI tumors | |
| Liposarcoma | Second most common of | Arises in fat cells in deep | Bears resemblance to fat |
| | STSs | tissue such as the inside of | cells when examined under |
| | | the thigh or in the | the microscope |
| | | retroperitoneum | |
| Leiomyosarcoma | Accounts for 5-10% | Arises in smooth muscle | Usually hemorrhagic, soft |
| (LMS) | STS cases | cells. Most common in the | and microscopically |
| | | uterus, stomach, small | pleomorphic, abundant |
| | | intestine and | mitotic figures |
| | | retroperitoneum | |
| Synovial sarcoma | Occurs most commonly | Occurs hear joints of the | Most SS are associated with |
| (SS) | in the young | arm, neck or leg | a reciprocal translocation |
| | | | t(x; 18)(p11.2; q11.2) |
| Malignant peripheral | Most common in the | Arises from the soft tissue | ~50% MPNST cases |
| nerve sheath tumors | young | surrounding nerves. Most | associated along with |
| (MPNST) | | arises from the nerve | neurofibromatosis type 1 |
| | | plexuses | (NF1), caused by a mutation |
| | | | in NF1 tumor suppressor |
| Rhabdomyosarcoma | Most commonly seen in | Arises from skeletal muscle | Diagnosis depends on |
| (RMS) | children aged 1-5. Most | progenitors. Can also be | recognition of |
| | common STS in | found attached to muscle | differentiation toward |
| | children, | tissue or wrapped around | skeletal muscle cells. |
| | | the intestine | myoD1 and myogenin used |
| | | | in diagnostic IHC tests |

Typically STS cases are sporadic, but germline mutations observed in a number of genes have been shown to cause predisposition to developing STS, in particular at a young age. For example, individuals carrying mutations in the TP53 tumor suppressor gene (Li-Fraumeni syndrome, LFS) have a highly elevated risk (12-21%, vs. 0.0004% in the general population) for developing STS. I n addition, the mean age at which LFS patients first develop STS is much younger than in the case of sporadic STS. Similarly, patients diagnosed with familial adenomatous polyposis (AFP) syndrome, caused by germline mutations of the APC tumor suppressor gene, are characterized by an increased risk of developing desmoid tumors. Furthermore, approximately 50% of MPNST develop in patients carrying inherited deletions of the NF1 gene. More recently, a family with GISTs was tested positive for germline mutations in the c-KIT oncogene.

STS can be divided into two classes. One class is characterized by distinct genetic changes and relatively simple karyotypes, such as point mutations or single chromosomal aberrations. Observed aberrations include mutations in the KIT oncogene in GISTs and mutations found in TP53, KRAS and EGFR in lung adenocarcinomas. Most simple-karyotype STS harbor fusion genes resulting from recurrent chromosomal translocations. These fusion genes typically encode transcription factors and occasionally, growth-factor signaling molecules. Alveolar rhabdomyosarcoma (ARMS) is one of the best studied translocation-associated STS. The pathogenesis of most, if not all ARMS, is attributed to a translocation between regions on the long arms of chromosome 2 and 13 [t(2:13)(q35:q14)], resulting in the fusion between transcription factors PAX3 and FKHR. As another example, in synovial sarcoma, translocation of chromosome 18 and the X chromosome generates the SYT-SSX1/2 products. Downstream targets of these fusion transcription factors are poorly recognized, but it has been shown that activation of the stem cell factors EZH2, OCT4, SOX2 and NANOG could play an important role in translocation-induced sarcoma-genesis. The second genotypic class of STS is highlighted by substantially complex karyotypes and numerous non-recurrent genetic changes. This class of STS is represented by UPS, LMS, and sarcomas generally with highly dedifferentiated and pleomorphic characteristics. Fifty percent (50%) of patients with this class of STSs will experience distant metastases and face a bleak prognosis.

As used herein, “overall survival” (OS) refers to the percentage of people in a study or treatment group who are still alive for a certain period of time after they were diagnosed with or started treatment for a disease, such as cancer. The overall survival rate is often stated as a five-year survival rate, which is the percentage of people in a study or treatment group who are alive five years after their diagnosis or the start of treatment. The phrase “measuring the gene-expression levels” or “determining the gene-expression levels” as used herein refers to determining or quantifying RNA or proteins expressed by the gene or genes. The term “RNA” includes mRNA transcripts, and/or specific spliced variants of mRNA. The term “RNA product of the gene” as used herein refers to RNA transcripts transcribed from the gene and/or specific spliced variants. In some embodiments, mRNA is converted to cDNA before the gene expression levels are measured. In the case of “protein”, it refers to proteins translated from the RNA transcripts transcribed from the gene. The term “protein product of the gene” refers to proteins translated from RNA products of the gene. A number of methods can be used to detect or quantify the level of RNA products of the gene or genes within a sample, including microarrays, Real-Time PCR (RT-PCR; including quantitative RT-PCR), nuclease protection assays, RNA-sequencing, and Northern blot analyses. In one embodiment, the assay uses the APPLIED BIOSYSTEMS™ HT7900 fast Real-Time PCR system. In addition, a person skilled in the art will appreciate that a number of methods can be used to determine the amount of a protein product of a gene of the invention, including immunoassays such as Western blots, ELISA, and immunoprecipitation followed by SDS-PAGE and immunocytochemistry. In certain embodiments, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR).

A person skilled in the art will appreciate that a number of detection agents can be used to determine gene expression. For example, to detect RNA products of the biomarkers, probes, primers, complementary nucleotide sequences or nucleotide sequences that hybridize to the RNA products can be used. In another example, to detect cDNA products of the biomarkers, probes, primers, complementary nucleotide sequences or nucleotide sequences that hybridize to the cDNA products can be used. To detect protein products of the biomarkers, ligands or antibodies that specifically bind to the protein products can be used.

As used herein, the term “hybridize” refers to the sequence specific non-covalent binding interaction with a complementary nucleic acid. In an embodiment, the hybridization is under high stringency conditions. Appropriate stringency conditions which promote hybridization are known to those skilled in the art.

As used herein, the term “probe” and “primer” as used herein refers to a nucleic acid sequence that will hybridize to a nucleic acid target sequence. In one example, the probe and/or primer hybridizes to an RNA product of the gene or a nucleic acid sequence complementary thereof. In another example, the probe and/or primer hybridizes to a cDNA product. The length of probe or primer depends on the hybridizing conditions and the sequences of the probe or primer and nucleic acid target sequence. In one embodiment, the probe or primer is at least 8, 10, 15, 20, 25, 50, 75, 100, 150, 200, 250, 400, 500, or more nucleotides in length. Probes and/or primers may include one or more label. In certain embodiments, a label may be any substance capable of aiding a machine, detector, sensor, device, or enhanced or unenhanced human eye from differentiating a labeled composition from an unlabeled composition. Examples of labels include, but are not limited to: a radioactive isotope or chelate thereof, dye (fluorescent or non-fluorescent), stain, enzyme, or nonradioactive metal. Specific examples include, but are not limited to: fluorescein, biotin, digoxigenin, alkaline phosphates, biotin, streptavidin, ³H, ‘⁴C, ³²P, ³⁵S, or any other compound capable of emitting radiation, rhodamine, 4-(4’-dimethylamino-phenylazo)benzoic acid; 4-(4′-dimethylamino-phenylazo)sulfonic acid (sulfonyl chloride); 5((2-aminoethyl)-amino)-naphtalene-1-sulfonic acid; Psoralene derivatives, haptens, cyanines, acridines, fluorescent rhodol derivatives, cholesterol derivatives; ethylenediaminetetraaceticacid and derivatives thereof or any other compound that may be differentially detected. The label may also include one or more fluorescent dyes. Examples of dyes include, but are not limited to: CAL-Fluor Red 610, CAL-Fluor Orange 560, dR110, 5-FAM, 6FAM, dR6G, JOE, HEX, VIC, TET, dTAMRA, TAMRA, NED, dROX, PET, BHQ+, Gold540, and LIZ.

As used herein, a “sequence detection system” is any computational method in the art that can be used to analyze the results of a PCR reaction. One example, inter alia, is the APPLIED BIOSYSTEMSTM HT7900 fast Real-Time PCR system. In certain embodiments, gene expression can be analyzed using, e.g., direct DNA expression in microarray, Sanger sequencing analysis, Northern blot, the NANOSTRING® technology, serial analysis of gene expression (SAGE), RNA-seq, tissue microarray, or protein expression with immunohistochemistry or western blot technique. PCR generally involves the mixing of a nucleic acid sample, two or more primers that are designed to recognize the template DNA, a DNA polymerase, which may be a thermostable DNA polymerase such as Taq or Pfu, and deoxyribose nucleoside triphosphates (dNTP's). Reverse transcription PCR, quantitative reverse transcription PCR, and quantitative real time reverse transcription PCR are other specific examples of PCR. In real-time PCR analysis, additional reagents, methods, optical detection systems, and devices known in the art are used that allow a measurement of the magnitude of fluorescence in proportion to concentration of amplified DNA. In such analyses, incorporation of fluorescent dye into the amplified strands may be detected or measured. In an embodiment, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR). As used herein the terms “differentially expressed” or “differential expression” refer to a difference in the level of expression of the genes that can be assayed by measuring the level of expression of the products of the genes, such as the difference in level of messenger RNA transcript expressed (or converted cDNA) or proteins expressed of the genes. In an embodiment, the difference can be statistically significant. The term “difference in the level of expression” refers to an increase or decrease in the measurable expression level of a given gene as measured by the amount of messenger RNA transcript (or converted cDNA) and/or the amount of protein in a sample as compared with the measurable expression level of a given gene in a control, or control gene or genes in the same sample.

In another embodiment, the differential expression can be compared using the ratio of the level of expression of a given gene or genes as compared with the expression level of the given gene or genes of a control, wherein the ratio is not equal to 1.0. For example, an RNA, cDNA, or protein is differentially expressed if the ratio of the level of expression in a first sample as compared with a second sample is greater than or less than 1.0. For example, a ratio of greater than 1, 1.2, 1.5, 1.7, 2, 3, 3, 5, 10, 15, 20 or more, ora ratio less than 1, 0.8, 0.6, 0.4, 0.2, 0.1, 0.05, 0.001 or less. In yet another embodiment the differential expression is measured using p-value. For instance, when using p-value, a biomarker is identified as being differentially expressed as between a first sample and a second sample when the p-value is less than 0.1, less than 0.05, less than 0.01, less than 0.005, or less than 0.001.

References herein to the “same” level of biomarker indicate that the level of biomarker measured in each sample is identical (i.e. when compared to the selected reference). References herein to a “similar” level of biomarker indicate that levels are not identical but the difference between them is not statistically significant (i.e. the levels have comparable quantities). As used herein, the terms “control” and “standard” refer to a specific value that one can use to determine the value obtained from the sample. In one embodiment, a dataset may be obtained from samples from a group of subjects known to have a soft tissue sarcoma type or subtype. The expression data of the genes in the dataset can be used to create a control (standard) value that is used in testing samples from new subjects. In such an embodiment, the “control” or “standard” is a predetermined value for each gene or set of genes obtained from subjects with soft tissue sarcoma whose gene expression values and tumor types are known. In certain embodiments of the methods disclosed herein, non-limiting examples of control genes can include, but are not limited to, ABCC1, ACTB, GAPDH, RelA, STAT5B, and YY1AP1. In some embodiments, a control population may comprise healthy individuals, individuals with cancer, or a mixed population of individuals with or without cancer.

As used herein, the term “normal” when used with respect to a sample population refers to an individual or group of individuals that does/do not have a particular disease or condition (e.g., STS) and is also not suspected of having or being at risk for developing the disease or condition. The term “normal” is also used herein to qualify a biological specimen or sample (e.g., a biological fluid) isolated from a normal or healthy individual or subject (or group of such subjects), for example, a “normal control sample”. The “normal” level of expression of a marker is the level of expression of the marker in cells in a similar environment or response situation, in a patient not afflicted with cancer. A normal level of expression of a marker may also refer to the level of expression of a “reference sample”, (e.g., sample(s) from a healthy subject(s) not having the marker associated disease). A reference sample expression may be comprised of an expression level of one or more markers from a reference database. Alternatively, a “normal” level of expression of a marker is the level of expression of the marker in non-tumor cells in a similar environment or response situation from the same patient that the tumor is derived from.

As defined herein, the terms “gene-expression profile,” “GEP, ” or “gene-expression profile signature” is any combination of genes, the measured messenger RNA transcript expression levels, cDNA levels, or direct DNA expression levels, or immunohistochemistry levels of which can be used to distinguish between two biologically different corporal tissues and/or cells and/or cellular changes.

In certain embodiments, a gene-expression profile is comprised of the gene-expression levels of at least 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 genes or less. In an embodiment, the gene-expression profile is comprised of 36 genes. In certain embodiments, the genes selected are: ABCB1, ABCC1, ABCG2, ACTB, ALAS1, ANLN, ANXA1, AQP3, BAX, Bcl2, Bcl2L/Bcl-xl, BIRC5, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPAS, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, or ZWINT. In an embodiment, the gene set comprises: ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN. In some embodiments, the gene set further comprises control genes selected from: ABCC1, ACTB, GAPDH, RelA, STAT5B, and YY1AP1.

As defined herein, “predictive training set” means a cohort of STS tumors with known clinical outcome for local recurrence, distant metastasis, or both and known genetic expression profile, used to define/establish all other STS tumors, based upon the genetic expression profile of each, as a low-risk, class 1 tumor type or a high-risk, class 2 tumor type. Additionally, included in the predictive training set is the definition of “threshold points” points at which a classification of metastatic risk is determined, specific to each individual gene expression level.

As defined herein, “altered in a predictive manner” means changes in genetic expression profile that predict local recurrence, distant metastasis, metastatic risk, or predict overall survival. Predictive modeling risk assessment can be measured as: 1) a binary outcome having risk of metastasis or overall survival that is classified as low risk (e.g., termed Class 1 herein) vs. high risk (e.g., termed Class 2 herein); and/or 2) a linear outcome based upon a probability score from 0 to 1 that reflects the correlation of the genetic expression profile of a STS tumor with the genetic expression profile of the samples that comprise the training set used to predict risk outcome. Within the probability score range from 0 to 1, a probability score, for example, less than 0.5 reflects a tumor sample with a low risk of local recurrence, metastasis or death from disease, while a probability score, for example, greater than 0.5 reflects a tumor sample with a high risk of local recurrence, metastasis or death from disease. The increasing probability score from 0 to 1 reflects incrementally declining metastasis free survival. In an embodiment, the probability score is a bimodal, two-class analysis, wherein a patient having a value of between 0 and 0.499 is designated as class 1 (low risk) and a patient having a value of between 0.500 and 1.00 is designated as class 2 (high risk).

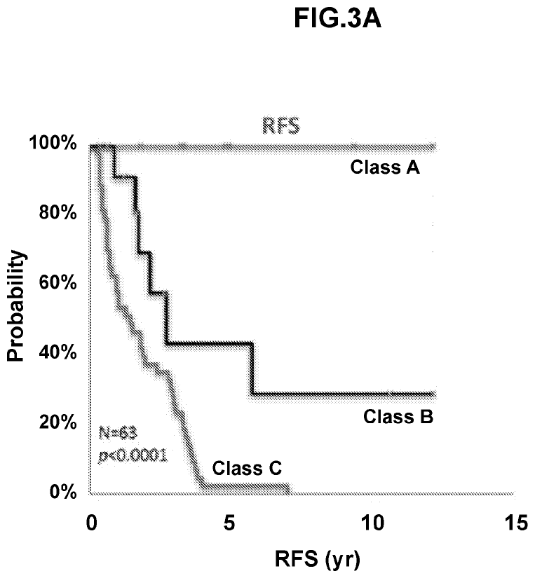

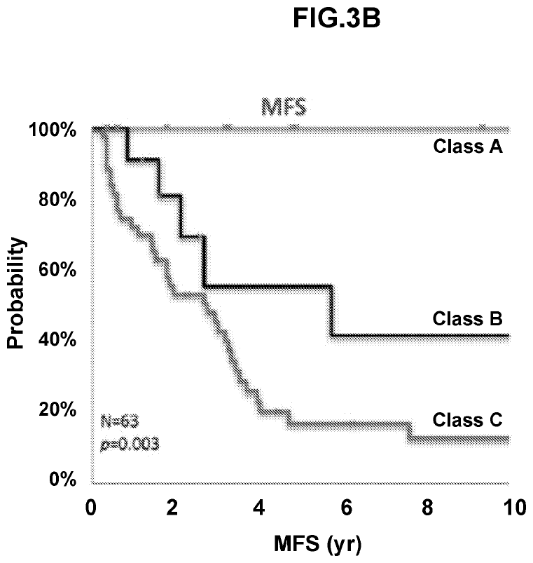

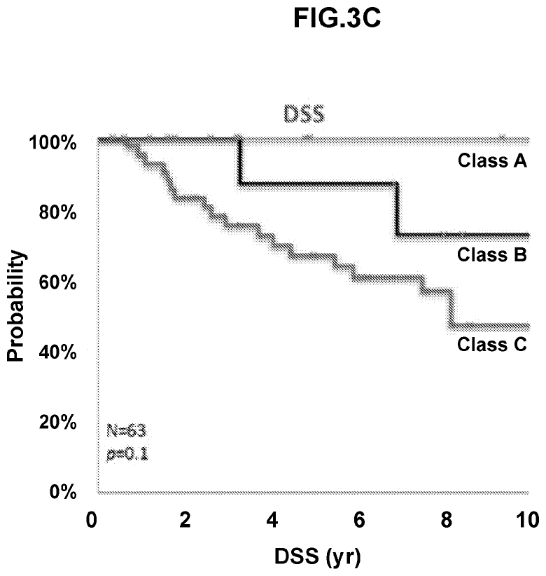

In certain embodiments, the probability score is a tri-modal, three-class analysis, wherein patients are designated as class A (low risk), class B (intermediate risk), or class C (high risk). To develop a ternary, or three-class system of risk assessment, with Class A having a low risk of metastasis or death from disease, Class B having an intermediate risk, and Class C having a high risk, the median probability score value for all low risk or high risk tumor samples in the training set was determined, and one standard deviation from the median was established as a numerical boundary to define low or high risk. For example, as shown in FIG. 3 and Table 10, low risk (Class A; with a probability score of 0-0.337) STS tumors within the ternary classification system have a 5-year metastasis free survival of 100%, compared to high risk (Class C; with a probability score of 0.673-1) tumors with a 17% 5-year metastasis free survival. Cases falling outside of one standard deviation from the median low or high risk probability scores have an intermediate risk, and intermediate risk (Class B; with a probability score of 0.338-0.672) tumors have a 55% 5-year metastasis free survival rate.

The TNM (Tumor-Node-Metastasis) status system is the most widely used cancer staging system among clinicians and is maintained by the American Joint Committee on Cancer (AJCC) and the International Union for Cancer Control (UICC). Cancer staging systems codify the extent of cancer to provide clinicians and patients with the means to quantify prognosis for individual patients and to compare groups of patients in clinical trials and who receive standard care around the world.

As defined herein, the term “aggressive cancer treatment regimen” is determined by a medical professional or team of medical professionals and can be specific to each patient. Whether a treatment is aggressive or not will generally depend on the cancer-type, the age of the patient, etc. For example, in breast cancer adjuvant chemotherapy is a common aggressive treatment given to complement the less aggressive standards of surgery and hormonal therapy. Those skilled in the art are familiar with various other aggressive and less aggressive treatments for each type of cancer. Advanced soft tissue sarcoma that is predicted to have an increased risk of recurrence, progression, or metastasis can be treated with an aggressive cancer treatment regimen. Advanced STS may be defined under two headings: (1) locoregional disease; and/or (2) distant metastases. Locoregional disease can be difficult to control and/or treat if: (1) the primary STS is located or involves a vital organ or structure that limits the potential for treatment; (2) recurrence after surgery or other therapy occurs because while likely not a result from metastasis, high rates of recurrence indicate an advanced STS tumor; and (3) presence of lymph node metastases, while rare in STS, indicate advanced disease. Distant metastases from a primary STS tumor can disseminate widely, and patients with distant metastases require aggressive treatments, which can eradicate metastatic sarcoma, prolong life and cure some patients. An aggressive cancer treatment regimen is defined by the National Comprehensive Cancer

Network (NCCN), and has been defined in the NCCN Guidelines® as including one or more of: 1) imaging (CT scan, PET/CT, MRI, chest X-ray), 2) discussion and/or offering of tumor resection if the tumor(s) is determined to be resectable, 3) radiation therapy, 4) chemoradiation, 5) chemotherapy, 6) regional limb therapy, 7) palliative surgery, 8) systemic therapy, 9) immunotherapy, and 10) inclusion in ongoing clinical trials. Guidelines for clinical practice are published in the National Comprehensive Cancer Network (NCCN Guidelines® Soft Tissue Sarcoma Version 2.2017 available on the World Wide Web at NCCN.org). Additional therapeutic options include, but are not limited to: 1) combination regimens such as: AD (doxorubicin, dacarbazine); AIM (doxorubicin, ifosfamide, mesna); MAID (mesna, doxorubicin, ifosfamide, dacarbazine); ifosfamide, epirubicin, mesna; gemcitabine and docetaxel; gemcitabine and vinorelbine; gemcitabine and dacarbazine; doxorubicin and olaratumab ; methotrexate and vinblastine; tamoxifen and sulindac; vincristine, dactinomycin, cylclophosphamide; vincristine, doxorubicin, cyclophosphamide; vincristine, doxorubicin, cyclophosphamide with ifosfamide and etoposide; vincristine, doxorubicin, ifosfamide; cyclophosphamide topotecan; ifosfamide, doxorubicin; and/or 2) single agents, such as, doxorubicin, ifosfamide, epirubicin, gemcitabine, dacarbazine, temozolomide, vinorelbine, eribulin, trabectedin, pazopanib, imatinib, sunitinib, regorafenib, sorafenib, nilotinib, dasatinib, interferon, toremifene, methotrexate, irinotecan, topotecan, paclitaxel, docetaxel, bevacizumab, temozolomide, sirolimus, everolimus, temsirolimus, crizotinib, ceritinib, palbociclib.

While surgical resection remains the mainstay for treating operable (Stage I-III) STS patients, for Stage I patients, en bloc resection with negative margins is generally considered sufficient for long-term local control. For those with incomplete resection margins and/or other unfavorable pathologic features, pre- or post-operative chemotherapy and/or radiation treatment can be recommended. No therapy has shown consistent efficacy for the treatment of resected STS, and treatment options for unresectable or advanced STS are limited. Targeted therapies have shown promising results in advanced/metastatic STS patients. For instance, the RTK (receptor tyrosine kinase) inhibitor pazopanib as a second line therapy extended progression-free survival (PFS) by three months for advanced non-lipogenic STS patients. In addition, mTOR inhibitors such as sirolimus, temsirolimus, and everolimus have also exhibited varying extent of effectiveness in patients with recurrent angiomyolipomas and lymphangioleiomyomatosis.

As used herein, the terms “treatment,” “treat,” or “treating” refers to a method of reducing the effects of a disease or condition or symptom of the disease or condition. Thus, in the disclosed methods, treatment can refer to a 5%, 10%, 20%, 30%, 40%, 50%, 60%, 70%, 80%, 90%, or 100% reduction in the severity of an established disease or condition or symptom of the disease or condition. For example, a method of treating a disease is considered to be a treatment if there is a 5% reduction in one or more symptoms of the disease in a subject as compared to a control. Thus, the reduction can be a 5%, 10%, 20%, 30%, 40%, 50%, 60%, 70%, 80%, 90%, 100% or any percent reduction between 5 and 100% as compared to native or control levels. It is understood that treatment does not necessarily refer to a cure or complete ablation of the disease, condition, or symptoms of the disease or condition. After a sarcoma is found and staged, a medical professional or team of medical professionals will recommend one or several treatment options. In determining a treatment plan, factors to consider include the type, location, and stage of the cancer, as well as the patient's overall physical health. Prior to the initiation of treatment and or therapy, all patients should be evaluated and managed by a multidisciplinary team with expertise and experience in sarcoma. Patients with sarcoma typically have a multidisciplinary health care team made up of doctors from different specialties, such as: an orthopedic surgeon (in particular, a surgeon who specializes in diseases of the bones, muscles, and joints), a surgical oncologist, a thoracic surgeon, a medical oncologist, a radiation oncologist, and/or a physiatrist (or rehabilitation doctor). After a sarcoma is found and staged, a medical professional or team of medical professionals will typically recommend one or several treatment options including one or more of surgery, radiation, chemotherapy, and targeted therapy.

In certain embodiments, the STS tumor is taken from a formalin-fixed, paraffin embedded sample. In another embodiment, the STS tumor is taken from image guided core biopsy, core needle biopsy, incisional biopsy, endoscope guided needle biopsy, endoscopic fine needle aspirate (EUS-FNA), or surgical biopsy.

In certain embodiments, analysis of genetic expression and determination of outcome is carried out using radial basis machine and/or partial least squares analysis (PLS), partition tree analysis, logistic regression analysis (LRA), K-nearest neighbor, or other algorithmic approach. These analysis techniques take into account the large number of samples required to generate a training set that will enable accurate prediction of outcomes as a result of cut-points established with an in-process training set or cut-points defined for non-algorithmic analysis, but that any number of linear and nonlinear approaches can produce a statistically significant and clinically significant result. As defined herein, “Kaplan-Meier survival analysis” is understood in the art to be also known as the product limit estimator, which is used to estimate the survival function from lifetime data. In medical research, it is often used to measure the fraction of patients living for a certain amount of time after treatment. JMP GENOMICS® software provides an interface for utilizing each of the predictive modeling methods disclosed herein, and should not limit the claims to methods performed only with JMP GENOMICS® software.

In another aspect, this disclosure relates to kits to be used in assessing the expression of a gene or set of genes in a STS sample or biological sample from a subject to assess the risk of developing recurrence, metastasis, or both. In an embodiment, the disclosure relates to a kit comprising primer pairs suitable for the detection and quantification of nucleic acid expression of at least ten genes selected from: ABCB1, ABCC1, ABCG2, ACTB, ALAS1, ANLN, ANXA1, AQP3, BAX, Bcl2, Bcl2L/Bcl-xl, BIRC5, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPA5, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT. In an embodiment of the kit, the primer pairs suitable for the detection and quantification of nucleic acid expression of at least ten genes are primer pairs for: ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN.

Kits can include any combination of components that facilitates the performance of an assay. A kit that facilitates assessing the expression of the gene or genes may include suitable nucleic acid-based and/or immunological reagents as well as suitable buffers, control reagents, and printed protocols. A “kit” is any article of manufacture (e.g. a package or container) comprising at least one reagent, e.g. a probe or primer set, for specifically detecting a marker or set of markers of the invention. The article of manufacture may be promoted, distributed, sold or offered for sale as a unit for performing the methods of the present invention. The reagents included in such a kit comprise probes/primers and/or antibodies for use in detecting one or more of the genes and/or gene sets disclosed herein and demonstrated to be useful for predicting recurrence, metastasis, or both, in patients with STS. Kits that facilitate nucleic acid based methods may further include one or more of the following: specific nucleic acids such as oligonucleotides, labeling reagents, enzymes including PCR amplification reagents such as Taq or Pfu, reverse transcriptase, or other, and/or reagents that facilitate hybridization. In addition, the kits of the present invention may preferably contain instructions which describe a suitable detection assay. Such kits can be conveniently used, e.g., in clinical settings, to diagnose and evaluate patients exhibiting symptoms of cancer, in particular patients exhibiting the possible presence of a soft tissue sarcoma.

### EXAMPLES

The Examples that follow are illustrative of specific embodiments of the invention, and various uses thereof. They are set forth for explanatory purposes only, and should not be construed as limiting the scope of the invention in any way.

### Materials and Methods

### Selection of Biomarkers for the GEP Discovery Set.

The inventors reviewed the literature for detailed reports and/or reviews on genetic expression of response and/or prognosis predictive markers, procedures of microarray analysis, and/or statistical data mining methods related to cancer in order to identify potential biomarkers for response and/or prognosis prediction in human cancers. Ninety-five (95) genes potentially related to mediation of chemoradiation response, cancer progression, cancer recurrence, or development of metastasis in human cancer types were chosen to be included in the “GEP discovery set” of 95 genes.

### STS Tumor Sample Preparation and RNA Isolation.

Formalin fixed paraffin embedded (FFPE) primary STS tumor specimens arranged in 5 μm sections on microscope slides were acquired under Institutional Review Board (IRB) approved protocols. All tissue was reviewed by a pathologist to confirm the presence of STS and the dissectible tumor area was marked. Tumor tissue was dissected from the slide using a sterile disposable scalpel, collected into a microcentrifuge tube, and deparaffinized using xylene. RNA was isolated from each specimen using the Ambion RECOVERALL™ Total Nucleic Acid Isolation Kit (Life Technologies Corporation, Grand Island, N.Y.). RNA quantity and quality were assessed using the NANODROP™ 1000 system and the Agilent Bioanalyzer 2100.

cDNA Generation and RT-PCR Analysis.

RNA isolated from FFPE samples was converted to cDNA using the APPLIED BIOSYSTEMS™ High Capacity cDNA Reverse Transcription Kit (Life Technologies Corporation, Grand Island, N.Y.). Prior to performing the RT-PCR assay each cDNA sample underwent a 14-cycle pre-amplification step. Pre-amplified cDNA samples were diluted 20-fold in TE buffer. 50 μ1 of each diluted sample was mixed with 50 μl of 2X TAQMAN® Gene Expression Master Mix, and the solution was loaded to a custom high throughput microfluidics gene card containing primers specific for the 95 genes. Each sample was run in duplicates. The gene expression profile test was performed on an APPLIED BIOSYSTEMSTM HT7900 machine (Life Technologies Corporation, Grand Island, N.Y.).

### Gene Expression Analysis.

Internal loading reference genes were determined by the geNorm program (qBASE+, Biogazelle, Belgium) based on minimal fluctuations of expression values across all STS cases. Mean Ct values were calculated from the average of the duplicates for each gene, and ΔCt values were obtained by subtracting the mean Ct from the geometric mean of the mean Ct values of all reference genes.

### Predictive Modeling and Cross Validation.

Prediction for risk of disease recurrence and prognosis was carried out by Partial Least Squares (PLS) predictive modeling using JMP Genomics V 7.0 (SAS v 9.4, CARY, N.C.). Area Under the Curve (AUC), accuracy, specificity, sensitivity, positive predictive value (PPV) and negative predictive value (NPV) were reported for each modeling test (JMP Genomics SAS). Cross validation analysis was carried out under various stratification strategies including 10-fold holdout, 5-fold holdout, and leave-3-out with 50 randomizations. Averaged/corrected error rate, AUC and specificity values were reported for cross validation studies.

### Survival Analysis.

Kaplan-Meier survival analysis and Cox univariate and multivariate regression analyses were performed in WinSTAT software (WinSTAT for Microsoft Excel, Version 2012.1) and JMP Genomics software (JMP, Cary, NC).

### Example 1

### Patient Demographics

Seventy-seven FFPE STS biopsy specimens from primary tumors were collected (Table 2).

The tumors ranged from stage I-IV, and leiomyosarcoma (LMS) was the primary tumor histotype in the cohort. Of samples evaluated, 55% had an RO resection margin, 13% had R1, and no gross tumor left by surgery (R2) was found. Sixty-three of 77 patients experienced disease recurrence, including local recurrence (n=20) or distant metastasis (43), and the remaining 14 patients were free of recurrence per the latest follow up visit. The endpoints of this current study were recurrence-free survival (locoregional, distant, or concurrent; RFS), metastasis-free survival (MFS; distant metastasis), and disease-specific survival (death within two years of the most recent distant metastatic event, DSS).

TABLE 2
Demographics of the 77 STS specimens evaluated.

| | Number | Percentage |
|---|---|---|
| Age | | |
| range | 34-91 | |
| median | 61 | |
| Gender | | |
| male | 27 | 35% |
| female | 50 | 65% |
| Location of primary | | |
| head and neck | 3 | 4% |
| thoracic or trunk | 11 | 14% |
| retro/intrabdominal | 35 | 45% |
| pelvic | 11 | 14% |
| upper extremity | 5 | 6% |
| lower extremity | 12 | 16% |
| Histotype | | |
| LMS | 46 | 60% |
| MFH/UPS | 18 | 23% |
| Other (NOS) | 13 | 17% |
| Stage | | |
| I | 7 | 9% |
| II | 18 | 23% |
| III | 35 | 45% |
| IV | 14 | 18% |
| UNK | 3 | 4% |
| Differentiation grade | | |
| 1 | 7 | 9% |
| 2 | 10 | 13% |
| 3/4 | 58 | 75% |
| UNK | 2 | 3% |
| Tumor size | | |
| ≤5 cm | 12 | 16% |
| >5 cm to ≤10 cm | 27 | 35% |
| >10 cm to ≤20 cm | 29 | 38% |
| >20 cm | 5 | 6% |
| UNK | 4 | 5% |
| Resection status (Stage I-III) | | |
| R0 | 42 | 55% |
| R1 | 10 | 13% |
| UNK | 25 | 32% |
| Progression status | | |
| Local recurrence | 20 | 26% |
| Metastasis | 53 | 69% |
| No progression | 14 | 18% |

### Example 2

### Gene Expression in STS

Expression levels of 95 candidate genes (Table 3) in the 77 STS specimens were determined using semi-quantitative RT-PCR analysis (AppliedBiosystems, Thermo Fisher Scientific). Average Ct values of all 95 genes were evaluated by geNorm program (qBASE+ software, Biogazelle Nev., Technologiepark 3, B-9052 Zwijnaarde, Belgium), and five genes among the 95 candidate genes with minimal changes in expression levels across all samples were selected as internal loading controls: V-Rel avian reticuloendotheliosis viral oncogene homolog (RelA), YY1 associated protein 1 (YY1AP1), ATP-binding cassette, sub-family C, member 1 (ABCC1), signal transducer and activator of transcription 5B (STAT5B), and actin beta (ACTB). The geometric mean (geomean) of the expression of the five control genes was calculated to represent the expression of controls. Expression of each of the remaining 90 genes was then normalized by subtracting the average Ct value of that gene from the geomean of the five controls. Five genes [cornulin (CRNN), Kallikrein-Related Peptidase 13 (KLK13), Lectin, Galactoside-Binding, Soluble, 7B (LGALS7B), Small Proline-Rich Protein 2C (SPRR2C), and Small Proline-Rich Protein 3 (SPRR3)] had undetectable expression in more than 75% of the cases in the cohort, and were excluded from the initial analysis.

TABLE 3
95 genes for the GEP discovery set.

| | | NCBI RefSeqID/ |
|---|---|---|
| Gene ID | Gene name | Accession No. |
| ABCB1 | ATP binding cassette subfamily B member 1 | NM_000927.4 |
| ABCC1 | ATP binding cassette subfamily C member 1 | NM_004996.3 |
| ABCG2 | ATP binding cassette subfamily G member 2 (Junior | NM_001257386.1 |
| | blood group) | |
| ACTB | actin beta | NM_001101.3 |
| ALAS1 | 5′-aminolevulinate synthase 1 | NM_000688.5 |
| ANLN | anillin, actin binding protein | NM_018685.4 |
| ANXA1 | Annexin A1 | NM_000700.2 |
| AQP3 | Aquaporin 3 | NM_004925.4 |
| BAX | BCL2-associated X protein | NM_004324.3 |
| Bcl2 | B-cell CLL/lymphoma 2 | NM_000633.2 |
| Bcl2L/Bcl-xl | BCL2-like 1 | NM_138578.2 |
| BIRC5 | baculoviral IAP repeat containing 5 | NM_001012270.1 |
| BMP4 | Bone morphagenic factor 4 | NM_001202.4 |
| CA9/CAIX | Carbonic Anhydrase IX | NM_001216.2 |
| CALD1 | Caldesmon | NM_004342.6 |
| CASP1 | Caspase1 | NM_001223.4 |
| CCL5 | C-C motif chemokine ligand 5 | NM_001278736.1 |
| CCND1 | Cyclin D1 | NM_053056.2 |
| CD44 | CD44 molecule | NM_000610.3 |
| CDC25B | cell division cycle 25b | NM_004358.4 |
| CDH1 | cadherin 1 | NM_004360.4 |
| CDK1 | cyclin dependent kinase 1 | NM_001170406.1 |
| CDKN1A | cyclin dependent kinase inhibitor 1A | NM_000389.4 |
| CDKN1B | cyclin dependent kinase inhibitor 1B | NM_004064.4 |
| CDKN2A | cyclin dependent kinase inhibitor 2A | NM_000077.4 |
| CFLAR | CASP8 and FADD-like apoptosis regulator | NM_001127183.2 |
| CLCA2 | chloride channel accessory 2 | NM_006536.5 |
| CRCT1 | cysteine rich C-terminal 1 | NM_019060.2 |
| CRNN | cornulin | NM_016190.2 |
| DPYD | Dihydropyrimidine dehydrogenase | NM_000110.3 |
| DSP | Desmoplakin | NM_001008844.2 |
| EGFR | epidermal growth factor receptor | NM_005228.3 |
| EPHA1 | EPH Receptor A1 | NM_005232.4 |
| EPHB3 | EPH Receptor B3 | NM_004443.3 |
| ERCC1 | ERCC excision repair 1, endonuclease non-catalytic | NM_001166049.1 |
| | subunit | |
| EZH1 | enhancer of zeste homolog 1 | NM_001991.3 |
| FGFR4 | Fibroblast growth factor receptor 4 | NM_002011.4 |
| FLT1 | fms-related tyrosine kinase 1 | NM_001159920.1 |
| GLI1 | GLI family zinc finger 1 | NM_001160045.1 |
| HIF1A | hypoxia inducible factor 1, alpha subunit | NP_001230013.1 |
| HSPA4 | heat shock protein family A (Hsp70) member 4 | NM_002154.3 |
| HSPA5 | heat shock protein family A (Hsp70) member 5 | NM_005347.4 |
| HSPB1 | heat shock protein family B (small) member 1 | NM_001540.3 |
| HSPD1 | heat shock protein family D (Hsp60) member 1 | NM_002156.4 |
| IGF1R | Insulin-Like Growth Factor 1 Receptor | NM_000875.4 |
| IVL | Involucrin | NM_005547.2 |
| KIT | KIT proto-oncogene receptor tyrosine kinase | NM_000222.2 |
| KLK13 | Kallikrein 13 | NM_015596.1 |
| LGALS7 | galectin 7 | NM_002307.3 |
| LYPD3 | LY6/PLAUR domain containing 3 | NM_014400.2 |
| MCM2 | minichromosome maintanance complex component 2 | NM_004526.3 |
| MITF | Microphthalmia-Associated Transcription Factor | NM_001184967.1 |
| MMP14 | matrix metallopeptidase 14 | NM_004995.3 |
| MMP2 | matrix metallopeptidase 2 | NM_001127891.2 |
| MMP9 | matrix metallopeptidase 9 | NM_004994.2 |
| MSH2 | mutS homolog 2 | NM_000251.2 |
| NFKB1A | NFKB inhibitor alpha | NM_020529.2 |
| PDCD4 | programmed cell death 4 (neoplastic transformation | NM_001199492.1 |
| | inhibitor) | |
| PDGFRA | platelet-derived growth factor receptor, alpha | NM_006206.4 |
| | polypeptide | |
| PERP | PERP, TP53 apoptosis effector | NM_022121.4 |
| PKP1 | Plakophilin 1 | NM_000299.3 |
| PLAUR | Plasminogen Activator, Urokinase Receptor | NM_001005376.2 |
| PTGS2 | prostaglandin-endoperoxide synthase 2 | NM_000963.3 |
| RELA/p65 | v-rel avian reticuloendotheliosis viral oncogene | NM_001145138.1 |
| | homolog A | |
| RELB | v-rel avian reticuloendotheliosis viral oncogene | NM_006509.3 |
| | homolog B | |
| S100A10 | S100 calcium binding protein A10 | NM_002966.2 |
| S100A2 | S100 calcium binding protein A2 | NM_005978.3 |
| SERPINE1 | Serpin Peptidase Inhibitor, Clade E (Nexin, | NM_000602.4 |
| | Plasminogen Activator Inhibitor | |
| SMAD 3 | SMAD family member 3 | NM_001145102.1 |
| SNAI1 | snail family transcriptional repressor 1 | NM_005985.3 |
| SNAI2 | snail family transcriptional repressor 2 | NM_003068.4 |
| SPARC | Secreted Protein, Acidic, Cysteine-Rich (Osteonectin) | NM_003118.3 |
| SPP1 | Osteoponin | NM_000582.2 |
| SPRR2C | small proline rich protein 2C (pseudogene) | NR_003062.1 |
| SPRR3 | small proline rich protein 3 | NM_005416.2 |
| STAT5B | signal transducer and activator of transcription 5B | NM_012448.3 |
| TGFB2 | transforming growth factor beta 2 | NM_001135599.2 |
| TGFBR2 | transforming growth factor beta receptor 2 | NM_001024847.2 |
| TIMP1 | TIMP metallopeptidase inhibitor 1 | NM_003254.2 |
| TIMP2 | TIMP metallopeptidase inhibitor 2 | NM_003255.4 |
| TNFRSF1A | tumor necrosis factor receptor superfamily, member | NM_001065.3 |
| | 1A | |
| TNFRSF1B | tumor necrosis factor receptor superfamily member | NM_001066.2 |
| | 1B | |
| TNFSF13 | tumor necrosis factor superfamily member 13 | NM_003808.3 |
| TRAF1 | TNF Receptor-Associated Factor 1 | NM_001190945.1 |
| TRIM29 | Tripartite motif-containing 29 | NM_012101.3 |
| TSPAN7 | tetraspanin 7 | NM_004615.3 |
| TWIST1 | twist basic helix-loop-helix transcription factor 1 | NM_000474.3 |
| TYMP | thymidine phosphorylase | NM_001113755.2 |
| TYMS | thymidylate synthetase | NM_001071.2 |
| VCAM1 | Vascular cell adhesion molecule 1 | NM_001078.3 |
| VEGFA | vascular endothelial growth factor A | NM_001025366.2 |
| YY1AP1 | YY1 Associated Protein 1 | NM_001198899.1 |
| ZFYVE9 | Zinc finger, FYVE domain containing 9 | NM_004799.3 |
| ZNF395 | Zinc finger protein 395 | NM_018660.2 |
| ZWINT | ZW10 interactor | NM_001005413.1 |

### Example 3

### Predictive Model Selection

Ten different predictive modeling algorithms were employed to evaluate gene expression in 63 STS specimens with stage I-III disease. Linear and non-linear models were compared for fitness to predict recurrence in the STS cohort using the expression of the 85 genes with sufficient expression data. A binary risk was assigned to each STS case based on evidence of recurrence, with “0” representing no recurrence (low risk, n=14) and “1” representing local and/or distant recurrence (high risk, n=49). Table 4 below shows the AUC, accuracy, specificity (identification of low risk cases) and sensitivity (identification of high risk cases) observed for each of the models assessed using JMP Genomics 7 (SAS 9.4). Partial least squares (PLS) was the most accurate model assessed, and was selected for subsequent downstream analyses.

TABLE 4
Comparison of accuracy among ten predictive models.

| Predictive model | AUC | Accuracy | Specificity | Sensitivity |
|---|---|---|---|---|
| Discriminant analysis | 0.78 | 0.78 | 0 | 0.98 |
| Distant scoring | 0.93 | 0.85 | 0.75 | 0.87 |
| General linear model | 0.97 | 0.92 | 0.67 | 0.98 |
| K-nearest neighbors | 0.67 | 0.76 | 0.33 | 0.87 |
| Logistic regression | 0.78 | 0.80 | 0.17 | 0.96 |
| Partial least squares | 0.99 | 0.98 | 1.0 | 0.98 |
| Partition trees | 0.91 | 0.93 | 0.75 | 0.98 |
| Quantile regression | 0.50 | 0.80 | 0 | 1.0 |
| Radial basis machine | 0.50 | 0.80 | 0 | 1.0 |
| Ridge regression | 0.93 | 0.80 | 0 | 1.0 |

### Example 4

### Discovery of a 36-gene GEP Signature for Recurrence Risk Prediction in STS

To identify subsets of the 95-gene GEP discovery set that are able to accurately predict recurrence, or distant metastasis, or both, a “variable importance value” (VIP) was generated by PLS as an indicator of the weight (significance) for each predictor variable (i.e. expression of gene) in the risk prediction process. The most significant 10, 20, 30, 36, and 40 genes as ranked by PLS were then tested for accuracy of recurrence prediction in the 63 STS cases. As shown in Table 5 below, adding six genes to the 30-gene set further augmented the AUC, accuracy and specificity of prediction. However, when four more genes were added to the 36-gene signature, accuracy and specificity for prediction dropped. Subsequent analyses were focused on the 36 most significant predictors modeled by PLS. A specificity of 0.92 and sensitivity of 0.98 could be translated into the ability of the 36 genes to correctly identify 11 of 12 low risk cases, and 46 of 47 high risk cases.

TABLE 5
Comparison of accuracy among the subsets of genes ranked by
significance of prediction by PLS.

| Gene set | AUC | Accuracy | Specificity | Sensitivity |
|---|---|---|---|---|
| Top 10 | 0.94 | 0.84 | 0.64 | 0.91 |
| Top 20 | 0.98 | 0.91 | 0.71 | 0.98 |
| Top 30 | 0.98 | 0.95 | 0.86 | 0.98 |
| Top 36 | 0.99 | 0.97 | 0.92 | 0.98 |
| Top 40 | 0.99 | 0.93 | 0.79 | 0.98 |

### Example 5

### Cross Validation Analysis

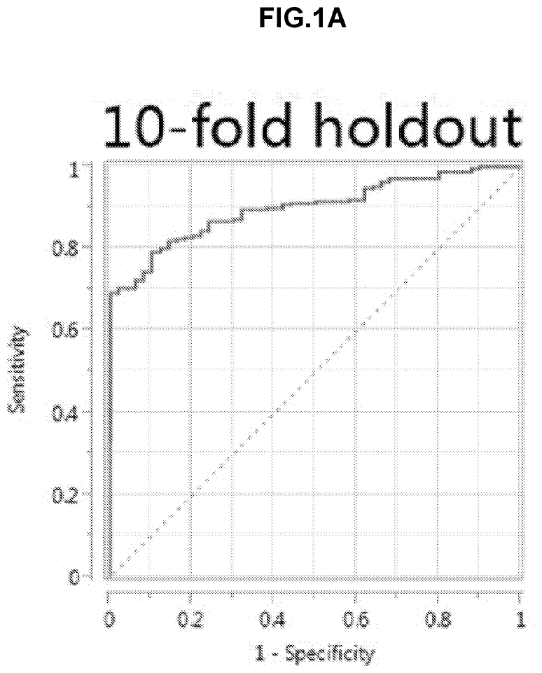

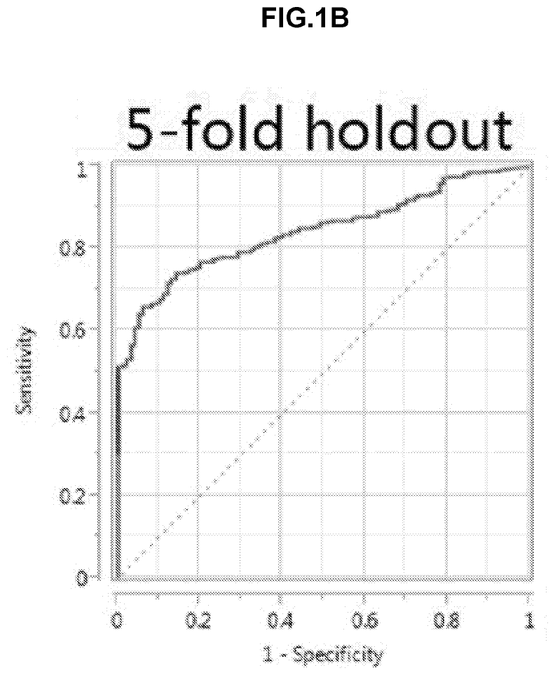

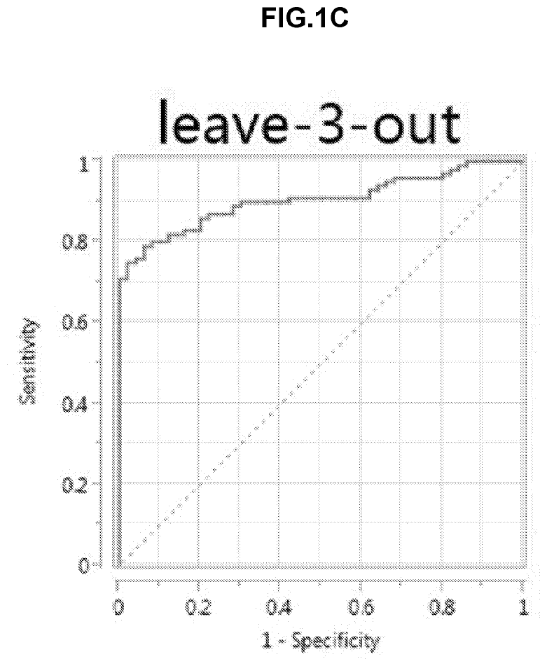

Cross validation (CV) analysis was performed to examine the fitness of the predictive model generated by the 36 genes using PLS. Three different CV methods were employed, including 10-fold, 5-fold, and leave-three out methods. Each method was performed with 50 iterations. All three CV methods generated average/corrected AUC of above or equal to 0.83 and accuracy above or equal to 77% (Table 6 and FIG. 1).

TABLE 6
Corrected root mean square error (RMSE), AUC, and accuracy
values generated by three cross validation analyses.

| | Average | | Average |
|---|---|---|---|
| CV method | RMSE | Average AUC | accuracy |
| 10-fold holdout | 0.35 | 0.84 | 0.78 |
| 5-fold holdout | 0.37 | 0.85 | 0.77 |
| Leave-3-out | 0.38 | 0.83 | 0.78 |

### Example 6

### Annotation of the 36-gene GEP

Table 7 shows the Gene ID, Gene Name, Cytoband, and expression levels of each of the 36 genes in non-recurrent and recurrent STS cases.

TABLE 7
List of genes for the STS 36-gene GEP.

| | | | Ex- | Ex- | | |
|---|---|---|---|---|---|---|
| | | | pression | pression | | |
| | | | in non-re- | in re- | | |
| | | | current | current | Relative | p |
| Gene ID | Gene name | Cytoband | STS | STS | expression | value |
| ABCB1 | ATP-Binding Cassette, Sub-Family B (MDR/TAP), Member 1 | 7q21.12 | −0.054 | 0.014 | 1.048 | 0.835 |
| ABCG2 | ATP-Binding Cassette, Sub-Family G (WHITE), Member 2 | 4q22 | 0.197 | −0.050 | 0.842 | 0.449 |
| AQP3 | Aquaporin 3 (Gill Blood Group) | 9p13 | −0.173 | 0.044 | 1.162 | 0.508 |
| BCL2 | B-cell CLL/lymphoma 2 | 18q21.3 | 0.357 | −0.091 | 0.733 | 0.168 |
| BCL2L1 | BCL2-Like 1 | 20q11.21 | 0.302 | −0.077 | 0.769 | 0.245 |
| CASP1 | caspase 1, apoptosis-related cysteine peptidase | 11q23 | 0.309 | −0.079 | 0.764 | 0.234 |
| CCL5 | chemokine (C-C motif) ligand 5 | 17q12 | 0.280 | −0.072 | 0.784 | 0.281 |
| CDH1 | cadherin 1, type 1, E-cadherin (epithelial) | 16q22.1 | 0.324 | −0.083 | 0.754 | 0.211 |
| CDK1 | cyclin-dependent kinase 1 | 10q21.2 | −0.445 | 0.114 | 1.473 | 0.084 |
| CDKN1A | cyclin-dependent kinase inhibitor 1A (p21, Cip1) | 6p21.1 | 0.652 | −0.166 | 0.567 | 0.010 |
| CRCT1 | Cysteine-Rich C-Terminal 1 | 1q21 | −0.210 | 0.054 | 1.201 | 0.419 |
| DSP | Desmoplakin | 6p24.3 | −0.144 | 0.037 | 1.133 | 0.581 |
| ERCC1 | excision repair cross-complementation group 1 | 19q13.32 | 0.318 | −0.081 | 0.758 | 0.220 |
| FGFR4 | Fibroblast growth factor receptor 4 | 5q35.2 | −0.155 | 0.040 | 1.144 | 0.552 |
| HSPD1 | heat shock 60 kDa protein 1 (chaperonin) | 2q33.1 | 0.226 | −0.058 | 0.821 | 0.384 |
| IGF1R | Insulin-Like Growth Factor 1 Receptor | 15q26.3 | 0.328 | −0.084 | 0.752 | 0.206 |
| LYPD3 | LY6/PLAUR domain containing 3 | 19q13.31 | 0.289 | −0.074 | 0.777 | 0.265 |
| MMP14 | matrix metallopeptidase 14 (membrane-inserted) | 14q11-q12 | −0.464 | 0.119 | 1.498 | 0.071 |
| MMP2 | matrix metallopeptidase 2 (gelatinase A, 72 kDa gelatinase) | 16q13-q21 | −0.331 | 0.084 | 1.333 | 0.202 |
| MSH2 | mutS homolog 2 | 2p21 | 0.287 | −0.073 | 0.779 | 0.269 |
| PDGFRA | platelet-derived growth factor receptor, alpha polypeptide | 4q12 | −0.297 | 0.076 | 1.294 | 0.253 |
| PKP1 | Plakophilin 1 | 1q32 | 0.287 | −0.073 | 0.779 | 0.268 |
| RELB | v-rel avian reticuloendotheliosis viral oncogene homolog B | 19q13.32 | −0.056 | 0.014 | 1.050 | 0.830 |
| SNAI1 | snail family zinc finger 1 | 20q13.2 | 0.158 | −0.040 | 0.871 | 0.544 |
| SNAI2 | snail family zinc finger 2 | 8q11.21 | 0.099 | −0.025 | 0.917 | 0.704 |
| SPARC | Secreted Protein, Acidic, Cysteine-Rich (Osteonectin) | 5q31-q33 | −0.172 | 0.044 | 1.162 | 0.508 |
| SPP1 | secreted phosphoprotein 1 | 4q22.1 | −0.289 | 0.074 | 1.286 | 0.265 |
| TIMP1 | TIMP metallopeptidase inhibitor 1 | Xp11.3- | −0.085 | 0.022 | 1.077 | 0.745 |
| | | p11.23 | | | | |
| TIMP2 | TIMP metallopeptidase inhibitor 2 | 17q25 | −0.487 | 0.124 | 1.528 | 0.058 |
| TNFRSF1A | tumor necrosis factor receptor superfamily, member 1A | 12p13.2 | −0.277 | 0.071 | 1.272 | 0.287 |
| TRAF1 | TNF Receptor-Associated Factor 1 | 9q33-q34 | 0.390 | −0.100 | 0.712 | 0.131 |
| TRIM29 | Tripartite motif-containing 29 | 11q23.3 | −0.692 | 0.177 | 1.825 | 0.006 |
| TYMS | Thymidylate Synthetase | 18p11.31- | 0.445 | −0.114 | 0.679 | 0.084 |
| | | p11.21 | | | | |
| VCAM1 | Vascular cell adhesion molecule 1 | 1p32-p31 | −0.180 | 0.046 | 1.170 | 0.489 |
| ZFYVE9 | Zinc finger, FYVE domain containing 9 | 1p32.3 | 0.271 | −0.069 | 0.790 | 0.296 |
| ZWINT | ZW10 interacting kinetochore protein | 10q21-q22 | −0.236 | 0.060 | 1.228 | 0.363 |

### Example 7

### Survival Analysis for GEP Predicted Risk Classes and Establishment of Normal and Reduced Confidence Intervals for Probability Scores

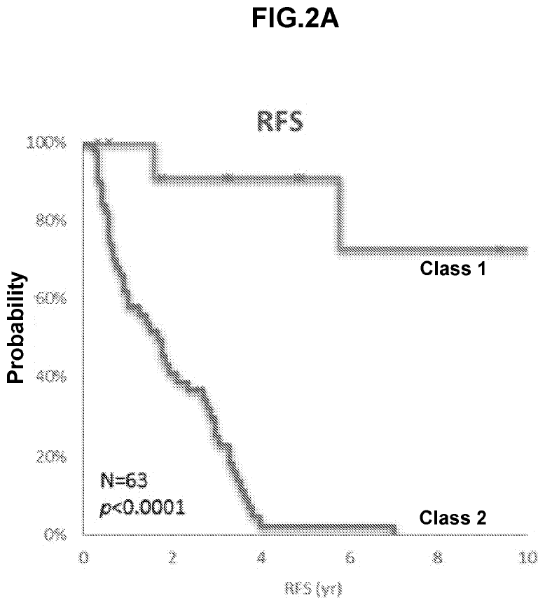

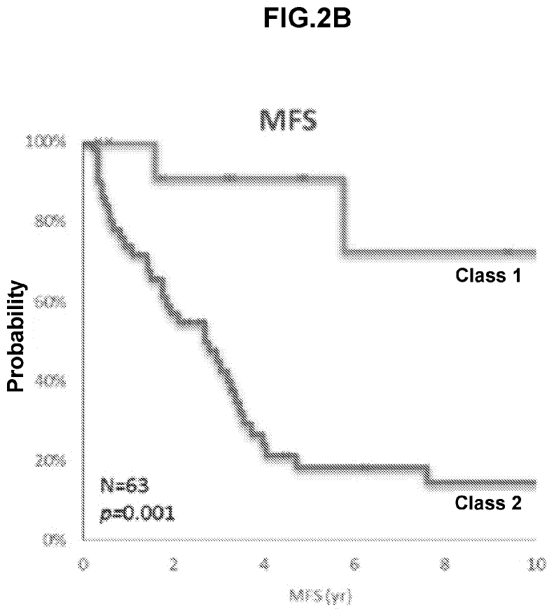

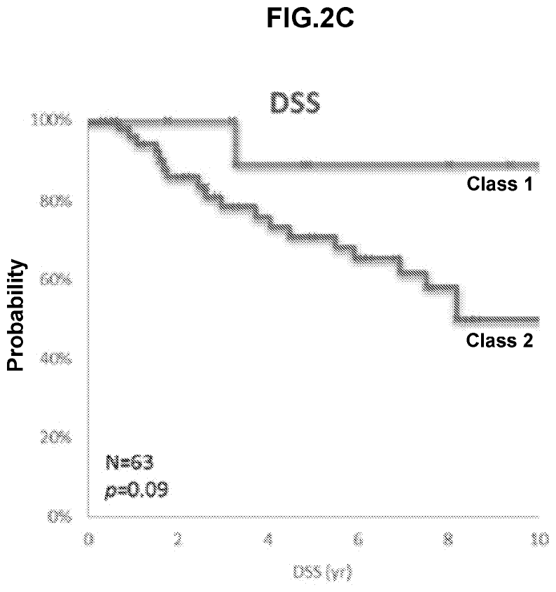

Kaplan-Meier survival analysis was performed to compare RFS, MFS, and DSS in the 36-gene GEP predicted class 1 and class 2 patients. As shown in FIG. 2A-2C and Table 8, class 1 and class 2 patients had highly stratified 5-year RFS and MFS (p<0.05), and DSS (p<0.09).

TABLE 8
Kaplan-Meier survival analysis comparing RFS, MFS, and DSS.

| | RFS | | MFS | | DSS | |
|---|---|---|---|---|---|---|
| | 5-year | # | 5-year | # | 5-year | # |
| | survival | events | survival | events | survival | events |
| Class 1 | 91% | 2 | 91% | 2 | 89% | 1 |
| (n = 13) | | | | | | |
| Class 2 | 2% | 47 | 18% | 37 | 71% | 19 |
| (n = 50) | | | | | | |

PLS predictive modeling algorithm provides a binary outcome of class 1 or class 2, along with a linear probability score that is indicative of how similar the gene profile of the analyzed sample is to the gene profiles of the samples in the training set. Probability score from 0-0.5 reflects a class 1 case, and a score from 0.5-1 indicates that the case will be predicted as class 2. Probability scores close to 0 and 1.0 suggest that the tumor's biology is in strong similarity to that of a defined class 1 and class 2 tumor, respectively. However, a score close to the 0.5 cutoff indicates that the tumor's genetics is less well defined as an established class 1 or class 2 case, therefore, class call could be ambiguous. To address this issue, a reduced confidence (RC) interval was established. Specifically, cases whose probability scores fall within one standard deviation (STDEV) of the mean probability score of the correctly predicted class 1 and class 2 from 0.5 were deemed to have RC for prediction, otherwise normal confidence (NC). In this cohort of 63 STS cases, class 1 and class 2 NC ranges are 0-0.337 (or Class A in a 3-tier risk class) and 0.673-1.0 (or Class C in a 3-tier risk class), respectively. Resultantly, a case with probability score between 0.338 and 0.672 falls into the RC interval (or Class B in a 3-tier risk class). Upon establishing the 3-tier risk classes, Kaplan-Meier survival analysis was again performed to compare RFS, MFS, and DSS for the 36-gene GEP predicted class 1 NC (Class A), RC (Class B), and class 2 NC (Class C). As shown in FIG. 3 and Table 9, when the probability score for binary risk prediction was set at 0.5, 13 patients had a class 1 prediction and 50 were predicted to be class 2 (FIG. 3A-3C).

TABLE 9
Kaplan-Meier survival analysis comparing RFS, MFS, and DSS
with reduced confidence (RC) interval.

| | RFS | | MFS | | DSS | |
|---|---|---|---|---|---|---|
| | 5-year | # | 5-year | # | 5-year | # |
| | survival | events | survival | events | survival | events |
| Class 1 | 100% | 0 | 100% | 0 | 100 | 0 |
| NC | | | | | | |
| (n = 7) | | | | | | |
| RC | 43% | 6 | 55% | 5 | 88% | 2 |
| (n = 13) | | | | | | |
| Class 2 | 2% | 43 | 17% | 34 | 67% | 18 |
| NC | | | | | | |
| (n = 43) | | | | | | |

### Example 8

### Comparison of RFS Predicted by GEP and Existing Clinical Factors

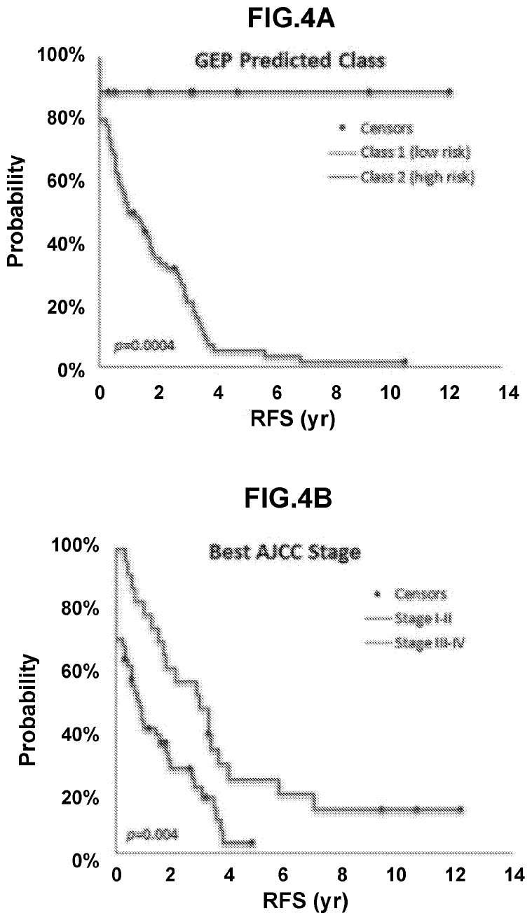

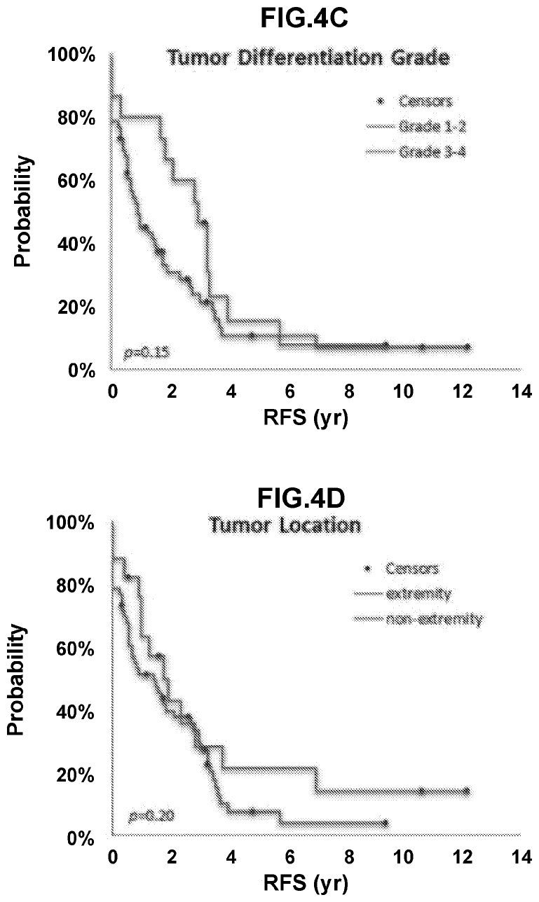

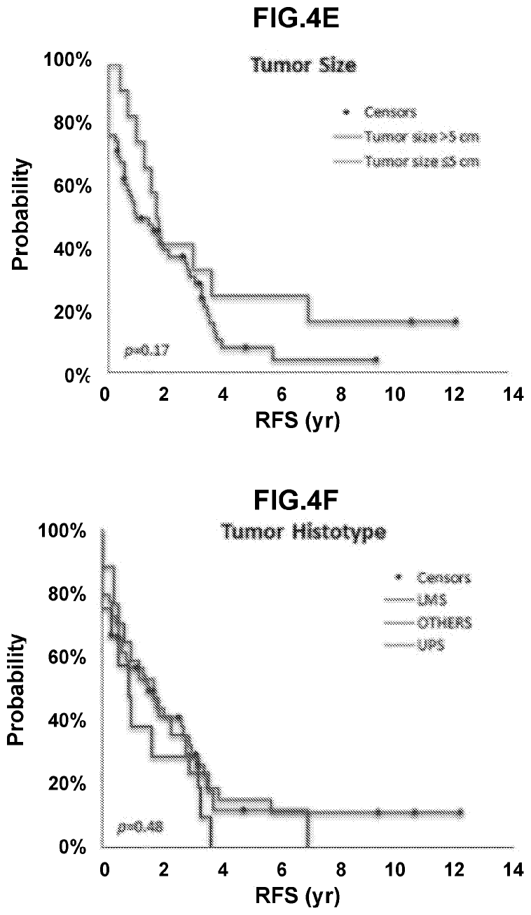

Kaplan-Meier survival analysis was performed to assess RFS in patient groups stratified according to GEP prediction (FIG. 4A) and conventional pathoclinical factors of STS of prognostic value, including diagnostic stage (FIG. 4B), tumor differentiation grade (FIG. 4C), location of primary tumor (extremity vs non-extremity) (FIG. 4D), size of tumor (5 cm cutoff) (FIG. 4E), and tumor histotype (LMS, UPS, or others) (FIG. 4F). As shown by the Kaplan-Meier survival curves, the 36-gene GEP predicted two risk classes had significantly more stratified RFS as compared to patients' clinical factors. Consistently, both univariate and multivariate Cox regression analyses demonstrated that only the 36-gene GEP class 1 and class 2 risk prediction, but none of the pathologic factors examined was an independent predictor for disease recurrence (Table 10). Five-year RFS rates for GEP predicted class 1 and class 2 patients were 100% and 2%, respectively. Ten-year RFS rates for the predicted low and high risk class patients were 75% and 0, respectively.

TABLE 10
Multivariate Cox regression analysis comparing GEP to
combined and individual staging factors to predict RFS.

| | | Lower 95% | Upper 95% | |
|---|---|---|---|---|
| Predictor | HR | CI | CI | p value |
| GEP (class 2 vs 1) | 28.30 | 26.28 | 30.31 | 0.001 |
| Location (non- vs extremity) | 1.82 | 0.96 | 2.67 | 0.17 |
| Stage (III vs I-II) | 1.02 | −0.43 | 2.48 | 0.97 |
| Grade (3-4 vs 1-2) | 1.66 | 0.32 | 2.99 | 0.46 |
| Tumor size (>5 cm vs ≤5 cm) | 1.07 | −0.32 | 2.46 | 0.93 |

### Example 9

### Comparison of MFS Predicted by GEP and Existing Clinical Factors

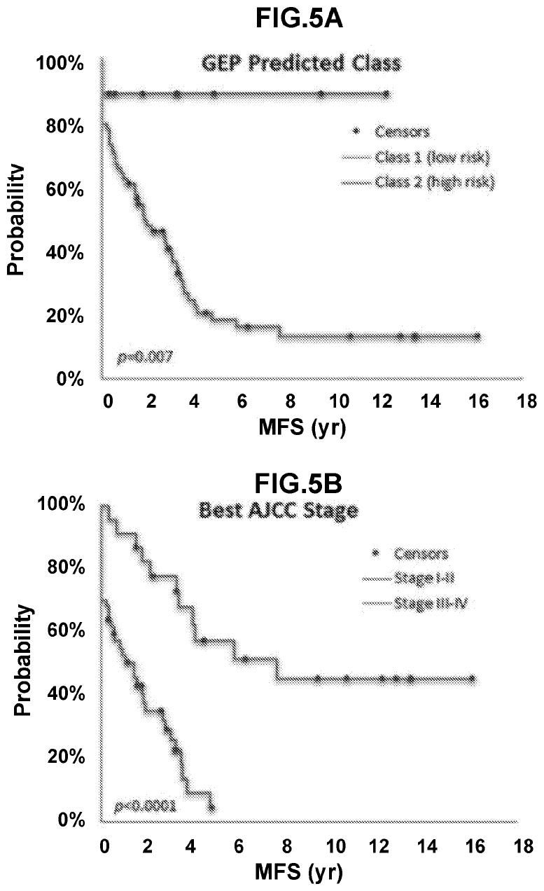

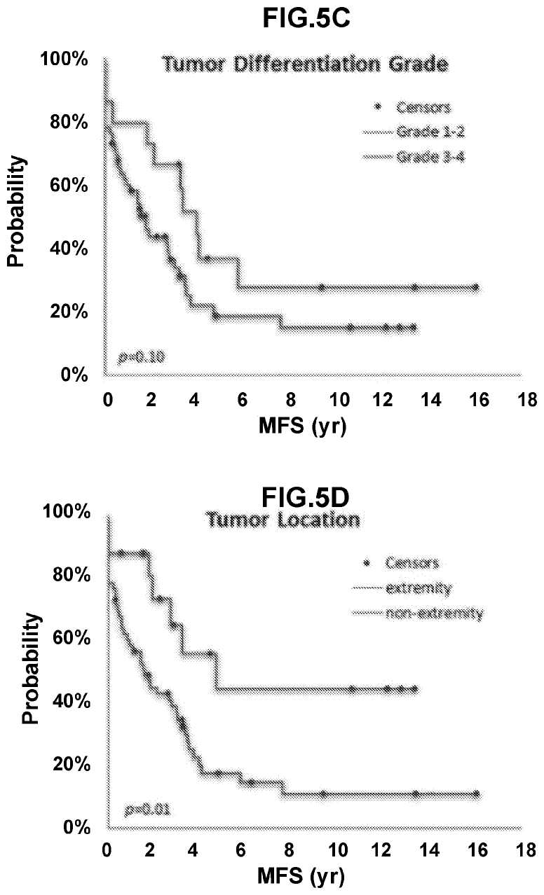

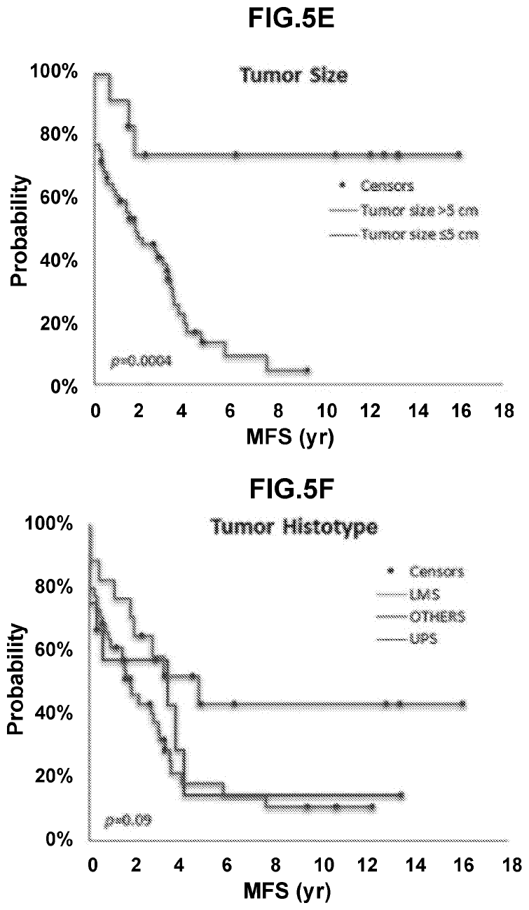

Kaplan-Meier and Cox regression analyses were performed on the 73 STS cases for the prediction of (distant) metastasis-free survival (FIG. 5). For the 36-gene GEP predicted low and high recurrence risk classes, five-year MFS rates were 100% and 18%, respectively, and ten-year MFS were 75% and 15%, respectively (FIG. 5A). Univariate Cox regression analysis indicated that GEP predicted high recurrence risk patients, tumor located at extremity, AJCC diagnostic Stage III, and tumor size exceeding 5 cm were all independent predictors of poor MFS. Multivariate Cox regression suggested that only GEP and tumor location were independent prognosticators for MFS (p<0.05), but GEP class 2 had a much higher hazard ratio (HR) as compared to tumor location at non-extremity site (Table 11.)

TABLE 11
Multivariate Cox regression analysis comparing GEP to
combined and individual staging factors to predict MFS.

| | | Lower 95% | Upper 95% | |
|---|---|---|---|---|
| Predictor | HR | CI | CI | p value |
| GEP (class 2 vs 1) | 14.80 | 12.79 | 16.82 | 0.01 |
| Location (non- vs extremity) | 3.52 | 2.44 | 4.61 | 0.02 |
| Stage (III vs I-II) | 2.56 | 1.41 | 3.70 | 0.11 |
| Grade (3-4 vs 1-2) | 1.14 | 0.04 | 2.24 | 0.82 |
| Tumor size (>5 cm vs ≤5 cm) | 2.63 | 1.24 | 4.01 | 0.17 |

### REFERENCES

BRAMWELL, “Management of advanced adult soft tissue sarcoma” Sarcoma, 2003. 7(5):p. 43-55.
CHIBON et al., Validated prediction of clinical outcome in sarcomas and multiple types of cancer on the basis of a gene expression signature related to genome complexity. Nat Med, 2010. 16(7): p. 781-7.
EILBER et al., Validation of the postoperative nomogram for 12-year sarcoma-specific mortality. Cancer, 2004. 101(10): p. 2270-5.
EILBER & KATTAN, Sarcoma nomogram: validation and a model to evaluate impact of therapy. J Am Coll Surg, 2007. 205(4 Suppl): p. S90-5.

KATTAN et al., A competing-risks nomogram for sarcoma-specific death following local recurrence. Stat Med, 2003. 22(22): p. 3515-25.
KATTAN et al., Postoperative nomogram for 12-year sarcoma-specific death. J Clin Oncol, 2002. 20(3): p. 791-6.
ITALIANO et al., Genetic profiling identifies two classes of soft-tissue leiomyosarcomas with distinct clinical characteristics. Clin Cancer Res, 2013. 19(5): p. 1190-6.
LAGARDE et al., Chromosome instability accounts for reverse metastatic outcomes of pediatric and adult synovial sarcomas. J Clin Oncol, 2013. 31(5): p. 608-15.
LUX et al., KIT extracellular and kinase domain mutations in gastrointestinal stromal tumors. Am J Pathol, 2000. 156(3): p. 791-5.
MARIANI et al., Validation and adaptation of a nomogram for predicting the survival of patients with extremity soft tissue sarcoma using a three-grade system. Cancer, 2005. 103(2): p. 402-8.
SILVEIRA et al., Genomic signatures predict poor outcome in undifferentiated pleomorphic sarcomas and leiomyosarcomas. PLoS One, 2013. 8(6): p. e67643.
von MEHREN, NCCN Clinical Practice Guidelines in Oncology Soft Tissue Sarcoma Version 1.2015. 2015.

1. A method for predicting risk of local recurrence, distant metastasis, or both, in a patient with a primary soft tissue sarcoma (STS) tumor, the method comprising:
(a) obtaining a STS tumor sample from the patient and isolating mRNA from the sample;
(b) determining the expression level of at least 10 genes in a gene set; wherein the at least ten genes in the gene set are selected from: ABCB1, ABCC1, ABCG2, ACTB, ALAS1, ANLN, ANXA1, AQP3, BAX, Bcl2, Bcl2L/Bcl-xl, BIRC5, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPA5, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT;
(c) comparing the expression levels of the at least 10 genes in the gene set from the STS tumor sample to the expression levels of the at least 10 genes in the gene set from a predictive training set to generate a probability score of the risk of local recurrence, distant metastasis, or both, and
(d) providing an indication as to whether the STS tumor has a low risk to a high risk of local recurrence, distant metastasis, or both, based on the probability score generated in step (c).

2. The method of claim 1, wherein the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR).

3. The method of claim 1, wherein the STS tumor sample is obtained from formalin-fixed, paraffin embedded sample.

4. The method of claim 1, wherein the probability score of local recurrence, distant metastasis, or both is between 0 and 1, and wherein a value of 1 indicates a higher probability of local recurrence, distant metastasis, or both, than a value of 0.

5. The method of claim 1, wherein the probability score is a bimodal, two-class analysis, wherein a patient having a value of between 0 and 0.499 is designated as class 1 (low risk) and a patient having a value of between 0.500 and 1.00 is designated as class 2 (high risk).

6. The method of claim 1, wherein the probability score is a tri-modal, three-class analysis, wherein patients are designated as class A (low risk), class B (intermediate risk), or class C (high risk).

7. The method of claim 1, further comprising identifying the STS tumor has a high risk of local recurrence, distant metastasis, or both, based on the probability score, and administering to the patient an aggressive tumor treatment.

8. The method of claim 1, wherein the gene set comprises the genes ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN.

9. The method of claim 8, wherein the gene set further comprises the genes ABCC1, ACTB, RelA, STAT5B, and YY1AP1.

10. A method for treating a patient with a primary soft tissue sarcoma (STS) tumor, the method comprising:
(a) obtaining a diagnosis identifying a risk of local recurrence, distant metastasis, or both, in a STS tumor sample from the patient, wherein the diagnosis was obtained by:
(1) determining the expression level of at least 10 genes in a gene set; wherein the at least 10 genes in the gene set are selected from: ABCB1, ABCC1, ABCG2, ACTB, ALAS1, ANLN, ANXA1, AQP3, BAX, Bcl2, Bcl2L/Bcl-xl, BIRCS, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPAS, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT;
(2) comparing the expression levels of the at least 10 genes in the gene set from the STS tumor sample to the expression levels of the at least 10 genes in the gene set from a predictive training set to generate a probability score of the risk of local recurrence, distant metastasis, or both, and;
(3) providing an indication as to whether the STS tumor has a low risk to a high risk of local recurrence, distant metastasis, or both, based on the probability score generated in step (2); and
(4) identifying that the STS tumor has a high risk of local recurrence, distant metastasis, or both, based on the probability score and diagnosing the STS tumor as having a high risk of local recurrence, distant metastasis, or both;
(b) administering to the patient an aggressive treatment when the determination is made in the affirmative that the patient has a STS tumor with a high risk of local recurrence, distant metastasis, or both.

11. The method of claim 10, further comprising performing a resection of the STS tumor when the determination is made in the affirmative that the patient has a STS tumor with a high risk of local recurrence, distant metastasis, or both.

12. The method of claim 10, wherein the expression level of each gene in a gene set is determined by reverse transcribing the isolated mRNA and measuring a level of fluorescence for each gene in the gene set by a nucleic acid sequence detection system following RT-PCR.

13. The method of claim 10, wherein the STS tumor sample is obtained from a formalin-fixed, paraffin embedded sample.

14. The method of claim 10, wherein the probability score is between 0 and 1, and wherein a value of 1 indicates a higher probability of local recurrence, distant metastasis, or both, than a value of 0.

15. The method of claim 10, wherein the probability score is a bimodal, two-class analysis, wherein a patient having a value of between 0 and 0.499 is designated as class 1 (low risk) and a patient having a value of between 0.500 and 1.00 is designated as class 2 (high risk).

16. The method of claim 10, wherein the probability score is a tri-modal, three-class analysis, wherein patients are designated as class A (low risk), class B (intermediate risk), or class C (high risk).

17. The method of claim 10, wherein the gene set comprises the genes ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN.

18. The method of claim 17, wherein the gene set further comprises the genes ABCC1, ACTB, RelA, STAT5B, and YY1AP1.

19. A method of treating a patient with a primary soft tissue sarcoma (STS) tumor, the method comprising administering an aggressive cancer treatment regimen to the patient, wherein the patient has a STS tumor with a probability score of between 0.500 and 1.00 as generated by comparing the expression levels of at least 10 genes selected from ABCB1, ABCC1, ABCG2, ACTB, ALAS1, ANLN, ANXA1, AQP3, BAX, Bcl2, Bcl2L/Bcl-xl, BIRC5, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPA5, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT from the STS tumor with the expression levels of the same at least ten genes selected from ABCB1, ABCC1, ABCG2, ACTB, ALAS1, ANLN, ANXA1, AQP3, BAX, Bcl2, Bcl2L/Bcl-xl, BIRC5, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPA5, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT from a predictive training set.

20. The method of claim 19, wherein the probability score is determined by a bimodal, two-class analysis, wherein a patient having a value of between 0 and 0.499 is designated as class 1 with a low risk of local recurrence, distant metastasis, or both, and a patient having a value of between 0.500 and 1.00 is designated as class 2 with an increased risk of local recurrence, distant metastasis, or both.

21. The method of claim 19, wherein the gene set comprises the genes ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN.

22. The method of claim 21, wherein the gene set further comprises the genes ABCC1, ACTB, RelA, STAT5B, and YY1AP1.

23. A kit comprising primer pairs suitable for the detection and quantification of nucleic acid expression of at least ten genes selected from: ABCB1, ABCC1, ABCG2, ACTB, ALAS1, ANLN, ANXA1, AQP3, BAX, Bcl2, Bcl2L/Bcl-xl, BIRCS, BMP4, CA9/CAIX, CALD1, CASP1, CCL5, CCND1, CD44, CDC25B, CDH1, CDK1, CDKN1A, CDKN1B, CDKN2A, CFLAR, CLCA2, CRCT1, CRNN, DPYD, DSP, EGFR, EPHA1, EPHB3, ERCC1, EZH1, FGFR4, FLT1, GLI1, HIF1A, HSPA4, HSPAS, HSPB1, HSPD1, IGF1R, IVL, KIT, KLK13, LGALS7, LYPD3, MCM2, MITF, MMP14, MMP2, MMP9, MSH2, NFKB1A, PDCD4, PDGFRA, PERP, PKP1, PLAUR, PTGS2, RELA/p65, RELB, S100A10, S100A2, SERPINE1, SMAD3, SNAI1, SNAI2, SPARC, SPP1, SPRR2C, SPRR3, STAT5B, TGFB2, TGFBR2, TIMP1, TIMP2, TNFRSF1A, TNFRSF1B, TNFSF13, TRAF1, TRIM29, TSPAN7, TWIST1, TYMP, TYMS, VCAM1, VEGFA, YY1AP1, ZFYVE9, ZNF395, and ZWINT.

24. The kit of claim 23, wherein the primer pairs suitable for the detection and quantification of nucleic acid expression of at least ten genes are primer pairs for: ABCB2, ABCG2, AQP3, BCL2, BCL2L1, CASP1, CCL5, CDH1, CDK1, CDKN1A, CRCT1, DSP, ERCC1, FGFR4, HSPD1, IGF1R, LYDP3, MMP14, MMP2, MSH2, PDGFRA, PKP1, RELB, SNAI1, SNAI2, SPARC, SPP1, TIMP1, TIMP2, TNFRSF1A, TRAF1, TRIM29, TYMS, VCAM1, ZFYVE9, and ZWTIN.

25. The kit of claim 24, wherein the primer pairs further comprise primer pairs for ABCC1, ACTB, RelA, STAT5B, and YY1AP1.
